(19) United States
(12) Patent Application Publication
(10) Pub. No.: US 2026/0130376 A1
(43) Pub. Date: May 14, 2026

(54) Bacillus Strains for Promoting Plant Health

(71) Applicant: CHR. HANSEN A/S, Hoersholm (DK)

(72) Inventors: Raquel AZEVEDO, Hoersholm (DK); Sailesh MALLA, Hoersholm (DK); Jan GERWIG, Hoersholm (DK); Cesar FONSECA, Hoersholm (DK); Ronnie MACHIELSEN, Hoersholm (DK)

(73) Assignee: CHR. HANSEN A/S, Hoersholm (DK)

(21) Appl. No.: 19/105,073

(22) PCT Filed: Aug. 29, 2023
(86) PCT No.: PCT/EP2023/073584

§ 371 Date: Feb. 20, 2025

(30) Foreign Application Priority Data
Aug. 30, 2022 (EP) 22192713.0

(51) Int. Cl. A01N 63/22; A01P 21/00; C07K 14/32; C12N 1/20; C12N 9/12; C12N 9/16; C12R 1/07

(52) U.S. Cl. CPC A01N 63/22; A01P 21/00; C07K 14/32; C12N 1/20; C12N 9/12; C12N 9/16; C12R 2001/07; C12Y 207/13003

(57) ABSTRACT

The present invention relates to provision of bacteria for promoting plant health under abiotic stress conditions, such as high salinity. In particular, the present invention relates to provision of bio-stimulant derivative Bacillus velezensis strains with improved root colonization properties for increasing plant growth.

### TECHNICAL FIELD OF THE INVENTION

The present invention relates to provision of bacteria for promoting plant health under abiotic stress conditions, such as high salinity. In particular, the present invention relates to provision of derivative Bacillus velezensis strains with improved root colonization properties for increasing plant growth.

### BACKGROUND OF THE INVENTION

The rapid population growth combined with climate change create a big challenge for crop production and yield globally. In one hand, there is an increasing demand of agricultural yield while on the other hand various biotic and abiotic issues significantly reduce crop production.

Soil salinity is an abiotic stress that poses a great threat to agriculture. Major crop losses annually occur due to toxic salts in the soil, particularly sodium chloride (NaCl). About 20% of the world cultivated area and around 50% of the world irrigated lands are affected by soil salinity, causing significant abiotic stress to plants. The main cause of increased salinity in irrigated areas are due to soluble salts carried in the irrigation water, which remain in the soil after evaporation and transpiration of the water. Unless these salts are leached from the soil, after prolonged time they accumulate to levels that are inhibitory to plant growth. This will also result in degradation of the soil structure affecting water and root penetration. Typically, plants stressed with NaCl are characterized by slower growth, premature leaf senescence, reduced tillering and lower yield. Sodium ions (Na⁺) are particularly damaging in high cytosolic concentration in leaf cells since they intervene with metabolic processes, and in particular, photosynthesis. Hence, among abiotic stresses, soil salinity and drought are considered a major issue.

Plant and soil microbes interact to help each other for their growth and development as well as to maintain the terrestrial eco-system. Plants can also use these growth promoting microbes as allies to withstand abiotic stress. Notably, plant growth promoting bacteria (PGPR) may help plants to grow in a high salinity soil by various mechanisms such as i) providing compatible solutes/osmolytes to the plants ii) by improving biofilm formation and root colonization to trap water molecules inside the biofilms, and/or iii) by balancing K⁺ and Na⁺ ion ratio in the plants.

Growth promoting microbes include Bacillus which are Gram-positive bacteria characterized by having thick cell walls and the absence of outer membranes. Much of the cell wall of Gram-positive bacteria is composed of peptidoglycan. Gram-positive species are divided into groups according to their morphological and biochemical characteristics. The genus Bacillus is belonging to the group of sporulating bacteria. Bacterial spores are one of the most resilient cell types; they resist many environmental changes, withstand dry heat and certain chemical disinfectants and may persist for years on dry land.

Accordingly, plant growth promoting biostimulant Bacillus strains have been applied, both naturally and commercially, as biofertilizers and as plant protectants. However, creating Bacillus strains with phenotypes adapted for stimulating plant growth at high soil salinity is very challenging without thorough knowledge of the target gene(s) involved. Typically, a combination of strain mutagenesis, smart screening, careful selection of candidate strains is necessary to identify and isolate improved derivative strains with desired phenotypes.

Moreover, bacterial inoculants will only benefit plant health under field conditions if they are capable of colonizing the plant, more specifically the roots. An obstacle for efficient root colonization is the native microbial community found in the soil near the roots of plants is known as the rhizomicrobiome. The area closest to the root surface is called the rhizosphere and is inhabited by indigenous microorganisms from the soil and the seed that have adapted to live in synergy with the plant. Bacterial inoculants will consequently face intense competition from the native rhizomicrobiome.

Hence, it is a complex task to obtain Bacillus strains capable of promoting plant growth under high salinity conditions. Further development of such derivative Bacillus strains would come with significant economic savings and improve the ability to meet the increasing global demands for crop production as the world population grow and climate changes enhance abiotic stress on farmland.

Thus, there is an unmet need for supplying improved Bacillus strains to aid plants challenged by abiotic stress and improve their yield under sub-optimal growth conditions.

Hence, it would be advantageous to provide Bacillus strains capable of proliferating under abiotic stress conditions, such as high salinity or drought. Specifically, it would be advantageous to provide Bacillus strains with improved root colonization which efficiently promote plant health and growth under conditions of abiotic stress, such as high salinity or drought.

### SUMMARY OF THE INVENTION

The present invention relates to Bacillus strains that promote plant fitness under conditions of abiotic stress, such as high salinity. In particular, the present invention discloses derivatives of a parental Bacillus strain with improved plant root colonization. The derivative Bacillus strains are prepared through a dual step campaign. Firstly, a classical strain improvement approach is adopted by exposure of a parental Bacillus strain to increasing salt concentration followed by identification and selection of lead strains with improved salt tolerance. Secondly, lead strains are subjected to extended growth on LB growth medium enriched with glycerol and manganese (LBGM) to induce biofilm formation and make it possible to identify derivative strains with a desired phenotype. Accordingly, the present invention makes available improved Bacillus strains to improve yield of plants or crops under conditions of high salinity.

Thus, an object of the present invention relates to the provision of Bacillus strains with improved capacity for root colonization and promotion of plant health under abiotic stress.

In particular, it is an object of the present invention to provide derivative strains of Bacillus that increases yield of plants or crops under conditions of high salinity or drought.

Thus, an aspect of the present invention relates to a derivative Bacillus velezensis strain, or variant thereof, with increased salt tolerance and root colonization compared to a parental strain of Bacillus velezensis deposited as DSM34004 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

Another aspect of the present invention relates to a method for preparing a derivative Bacillus velezensis strain with increased salt tolerance and/or root colonization compared to a parental strain of Bacillus velezensis as described herein, said method comprising the steps of:
(i) providing a parental strain of Bacillus velezensis
(ii) growing said parental strain of Bacillus velezensis under conditions of high salinity to prepare a pool of derivative Bacillus velezensis strains comprising one or more mutations, and
(iii) selecting a derivative Bacillus velezensis strain with increased salt tolerance and/or root colonization from said pool of derivative Bacillus velezensis strains.

Yet another aspect of the present invention relates to a derivative Bacillus velezensis strain with increased salt tolerance and root colonization compared to a parental strain of Bacillus velezensis obtainable by the method as described herein.

Still another aspect of the present invention relates to a composition comprising the derivative Bacillus velezensis strain with increased salt tolerance and root colonization compared to a parental strain of Bacillus velezensis as described herein.

A further aspect of the present invention relates to a plant or seed coated with a composition as described herein.

A still further aspect of the present invention relates to a method of increasing resistance of a plant against a condition of abiotic stress, said method comprising administering a derivative Bacillus velezensis strain with increased salt tolerance and root colonization compared to a parental strain of Bacillus velezensis as described herein or a composition as described herein to the plant, to a part of the plant and/or to the habitat of the plant.

An even further aspect of the present invention relates to use of a derivative Bacillus velezensis strain with increased salt tolerance and root colonization compared to a parental strain of Bacillus velezensis as described herein or a composition as described herein as a biostimulant, a growth promoter and/or to increase resistance of a plant against a condition of abiotic stress.

Yet another aspect of the present invention relates to a kit comprising:
(i) a derivative Bacillus velezensis strain with increased salt tolerance and root colonization compared to a parental strain of Bacillus velezensis, a composition or coated plant as described herein;
(ii) a container; and
(iii) optionally, instructions for use.

### BRIEF DESCRIPTION OF THE FIGURES

FIG. 1 shows a biofilm formation of the parental strain S-P and lead candidate derivative Bacillus strains S1-S10 on LBGM plates.

FIG. 2 shows growth of lead candidate derivative Bacillus strains S1-S7 and parental strain S-P in CSE medium supplemented with 1 M NaCl in the Growth Profiler setup.

FIG. 3 shows root colonization of 7-days old Arabidopsis thaliana seedlings inoculated with the parental strain S-P and selected lead candidate derivative Bacillus strains at 24 hours after inoculation. (A) representative figures of inoculated seedlings at 24 h. Arrows indicate the root and differences in thickness of the root are visible between the parental strain and the derivative Bacillus strains. (B) Colony forming units (CFUs) present at 24 hours after inoculation in the growth medium and attached to Arabidopsis seedling roots. (C) Ratio of attached cells present in the seedling root at 24 hours after inoculation over the total bacterial cells.

The present invention will in the following be described in more detail.

### DETAILED DESCRIPTION OF THE INVENTION

### Definitions

Prior to outlining the present invention in more details, a set of terms and conventions is first defined:

### Plant Biostimulant

In the present context, the term “plant biostimulant” refers to any substance or microorganism applied to plants with the ability to enhance nutrition efficiency, abiotic stress tolerance and/or crop quality traits, regardless of its nutrients content. By extension, plant biostimulants also designate commercial products containing mixtures of such substances and/or microorganisms.

### Plant Growth Promoting Agent

In the present context, the term “plant growth promoting agent” or “plant growth promoting microorganism” refers to a microorganism with the ability to colonize roots and/or inner plant tissues and promote plant growth and health by either acting as a biofertilizer, biostimulant or via biological control of plant disease.

### Parental Strain

In the present context, the term “parental strain” refers to a microorganism that is the origin to one or more derived strains, i.e. it is designating the first generation giving rise to one or more succeeding generation.

### Derivative Strain

In the present context, the term “derivative strain” refers to a microorganism that is a second generation derived from a parental strain. The derivative strain may be developed by mutagenesis, wherein one or more mutations are introduced into the genome of the parental strain. The mutations may be introduced by adaptive laboratory evolution by exposing the parental strain to increasing concentrations of salt, such as NaCl.

### Identifying Characteristics

In the present context, the term “identifying characteristics” refers to the phenotype of a microorganism, i.e. the set of observable characteristics or traits of the microorganism. Particularly, the identifying characteristic can be increased salt tolerance.

Microorganisms sharing all identifying characteristics can have different non-identical genomic sequences. This may be the case if mutations are silent or conservative, i.e. the new codon gives rise to the same amino acid or the new amino acid have similar biochemical properties (e.g. charge or hydrophobicity), respectively.

### Salt Tolerance

In the present context, the term “salt tolerance” refers to the concentration of salt, such as NaCl, needed to inhibit the proliferation of a cell population, such as a Bacillus strain, by 50%. This is also called the half maximal inhibitory concentration (IC50).

“Salt tolerance” may be measured with respect to typical salts found in irrigation water including sodium chloride (NaCl), sodium sulphate (Na₂SO₄), sodium bicarbonate (NaHCO₃), magnesium sulphate (MgSO₄), calcium sulphate (CaSO₄), calcium chloride (CaCl₂)), potassium chloride (KCl), and potassium sulphate (K₂SO₄).

Preferably, “salt tolerance” is measured with respect to NaCl.

### Root Colonization

In the present context, the term “root colonization” refers to the ability of bacteria to populate the roots of a plant. Root colonization is promoted by the ability of the Bacillus strain to form biofilm as it will enhance the ability of the bacterial inoculant to compete with and/or modulate the native microbial community of the rhizosphere (i.e. the rhizomicrobiome).

Root colonization of a Bacillus strain may be determined by growing plant seedlings in vitro and measuring the fraction of cells attached to the roots after a period of incubation (e.g. 24 hours after inoculation of 10⁵ cells). Root colonization is quantified as the ratio of cells attached to the seedling roots to the total number of bacterial cells, measured as colony forming units (CFU). To quantify the cells attached to the roots, seedlings are sonicated to release the cells into suspension.

### Biofilm

In the present context, the term “biofilm” refers to a population of bacteria that stick to each other via a slimy extracellular matrix. The components of the extracellular matrix are produced by the cells in the biofilm and include polysaccharides, proteins, lipids, and extracellular DNA. Biofilms share some of the characteristics of a hydrogel in that they can store many times its dry weight in water.

Biofilm formation of the Bacillus strain is advantageous because it improves the ability of the bacteria to adhere to and colonize the roots of plants and shields the bacteria when conditions for proliferation are challenging. Moreover, biofilms protect plants from abiotic stress by trapping water and thereby mitigating the effect of any abiotic stress, such as elevated salt concentrations or drought.

### Variant or Variant Strain

In the present context, the term “variant” or “variant strain” refers to a strain which is functionally equivalent to a strain of the invention, e.g. having substantially the same, or improved, properties (e.g. regarding salt tolerance). Such variants, which may be identified using appropriate screening techniques, are a part of the present invention.

The term “variant” or “variant strain” is a strain derived, or a strain which can be derived, from a strain of the invention by means of mutagenesis e.g. genetic engineering, adaptive laboratory evolution, physical and/or chemical treatment.

Especially, the term “variant” or “variant strain” refers to a strain obtained by subjecting a strain of the invention to any conventionally used mutagenesis treatment including classical strain improvement, treatment with a chemical mutagen such as ethane methane sulphonate (EMS) or N-methyl-N′-nitro-N-nitroguanidine (NTG), UV light, or to a spontaneously occurring mutant. A variant or variant strain may have been subjected to several mutagenesis treatments (a single treatment should be understood as one mutagenesis step followed by a screening/selection step), but it is presently preferred that no more than 20, or no more than 10, or no more than 5, treatments (or screening/selection steps) are carried out.

In a presently preferred variant or variant strain, less than 5%, or less than 1% or even less than 0.1% of the nucleotides in the bacterial genome have been modified with another nucleotide, or deleted, compared to the parental strain.

The terms “variant” and “variant strain” are used interchangeably herein.

### Nomenclature of Mutation

In the present context, the conventional one-letter and three-letter codes for amino acid residues are used. For ease of reference, amino acid changes in variants of the invention are described by use of the following nomenclature: amino acid residue in the parent enzyme; position; substituted amino acid residue(s).

According to this nomenclature, the substitution of, for instance, an alanine residue for a glycine residue at position 20 is indicated as Ala20Gly or A20G. The deletion of alanine in the same position is shown as Ala20* or A20*. The insertion of an additional amino acid residue (e.g. a glycine) is indicated as Ala20AlaGly or A20AG. The deletion of a consecutive stretch of amino acid residues (e.g. between alanine at position 20 and glycine at position 21) is indicated as DELTA (Ala20-Gly21) or DELTA (A20-G21). When a parent enzyme sequence contains a deletion in comparison to the enzyme sequence used for numbering an insertion in such a position (e.g. an alanine in the deleted position 20) is indicated as *20Ala or *20A. Multiple mutations are separated by a plus sign or a slash. For example, two mutations in positions 20 and 21 substituting alanine and glutamic acid for glycine and serine, respectively, are indicated as A20G+E21S or A20G/E21S. When an amino acid residue at a given position is substituted with two or more alternative amino acid residues these residues are separated by a comma or a slash. For example, substitution of alanine at position 30 with either glycine or glutamic acid is indicated as A20G,E or A20G/E, or A20G, A20E.

When a position suitable for modification is identified herein without any specific modification being suggested, it is to be understood that any amino acid residue may be substituted for the amino acid residue present in the position. Thus, for instance, when a modification of an alanine in position 20 is mentioned but not specified, it is to be understood that the alanine may be deleted or substituted for any other amino acid residue (i.e. any one of R, N, D, C, Q, E, G, H, I, L, K, M, F, P, S, T, W, Y, V).

### Mutation

In the present context, the term “mutation” refers to an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift.

A deletion is to be understood as a genetic mutation resulting in the removal of one or more nucleotides of a nucleotide sequence of the genome of an organism; an insertion is to be understood as the addition of one or more nucleotides to the nucleotide sequence; a substitution (or point mutation) is to be understood as a genetic mutation where a nucleotide of a nucleotide sequence is substituted by another nucleotide; a frameshift is to be understood as a genetic mutation caused by a insertion or deletion of a number of nucleotides in a nucleotide sequence that is not divisible by three, therefore changing the reading frame and resulting in a completely different translation from the original reading frame; an introduction of a stop codon is to be understood as a point mutation in the DNA sequence resulting in a premature stop codon; an inhibition of substrate binding of the encoded protein is to be understood as any mutation in the nucleotide sequence that leads to a change in the protein sequence responsible for preventing binding of a substrate to its catalytic site of the protein.

Furthermore, a knockout mutant is to be understood as genetic mutation resulting in the removal or deletion of a gene, such as an entire gene or an entire open reading frame from the genome of an organism.

In the present description and claims the conventional one-letter code for nucleotides is used following the analogous principles as described for amino acids nomenclature supra.

### About

Wherever the term “about” is employed herein in the context of amounts, for example absolute amounts, such as numbers, purities, concentrations, weights, sizes, etc., or relative amounts (e.g. percentages, equivalents, fractions or ratios), timeframes, and parameters such as temperatures, pressure, etc., it will be appreciated that such variables are approximate and as such may vary by ±10%, for example±5% and preferably ±2% (e.g. ±1%) from the actual numbers specified. This is the case even if such numbers are presented as percentages in the first place (for example ‘about 10%’ may mean±10% about the number 10, which is anything between 9% and 11%).

### Sequence Identity

In the present context, the term “sequence identity” is here defined as the sequence identity between proteins at the amino acid level. The protein sequence identity may be determined by comparing the amino acid sequence in a given position in each sequence when the sequences are aligned.

To determine the percent identity of two amino acid sequences, the sequences are aligned for optimal comparison purposes (e.g. gaps may be introduced in the sequence of a first amino acid sequence for optimal alignment with a second amino acid sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100).

In one embodiment, the two sequences are the same length. In another embodiment, the two sequences are of different length and gaps are seen as different positions.

One may manually align the sequences and count the number of identical amino acids. Alternatively, alignment of two sequences for the determination of percent identity may be accomplished using a mathematical algorithm. Such an algorithm is incorporated into the XBLAST program of (Altschul et al. 1990). BLAST protein searches may be performed with the XBLAST program, score=50, wordlength=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilizing the XBLAST and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment.

The percent identity between two sequences may be determined using techniques similar to those described above, with or without allowing gaps. In calculating percent identity, only exact matches are counted.

### Derivative Bacillus Strains

Agriculture is globally faced with the challenge of maximizing agricultural yield to meet the steep global demands of large quantities of plants or crops produced under conditions that are becoming gradually more challenging. Specifically, abiotic stress, such as high salinity and drought, are increasingly more frequent events caused by e.g. climate changes and/or intensive irrigated farming.

A source of salinity issues is irrigation water, wherein salts provided with the irrigation water accumulate in the soil over time to a point at which more salt is provided than the plants can uptake. Salt may leach out of the soil with irrigation water or rainwater moving materials, including salt and organic material, downward through the soil. However, leaching may be inhibited by high clay content or compaction of the soil, wherein water remains at the surface and evaporates, depositing the salt content but not leaching undissolved salts below the root zone. Problems with high salinity is therefore compounded in arid or semi-arid regions or under conditions of drought. If the salt concentration in the soil becomes greater than the salt concentration in the plant, there will be a net movement of water from the plant into the soil, which will inhibit plant growth or ultimately lead to plant death.

There are strategies to mitigate the negative effect of high salinity soils in farmlands. These include improved drainage, leaching (e.g. by increased watering with salt-deficient water), and reducing surface water evaporation. However, none of these strategies eliminate the challenges with high salinity in the soil. Notably, tillage is a non-permanent treatment of the soil that may temporarily increase drainage, but with the soil likely re-sealing. Leaching is only effective if the soil drains well and even then, it is a costly and non-environmentally friendly procedure that requires application of huge amount of water. Evaporation can be reduced by covering the soil with residue or mulch but comes with economic drawbacks and the risk of creating an anaerobic environment that facilitates fungal diseases to develop at plant stems and/or roots. Therefore, solutions to enable cultivation under abiotic stress, such as high salinity or drought, are in demand.

Bacillus strains can be used in biological agriculture with different benefits depending on the bacterial strain used. Biological agriculture or food industries can rely on natural methods of classical strain improvement (CSI) techniques to create improved strains and products. This approach is guided by introduction of random mutations to a parental strain followed by screening and selection of improved variants. Mutations of classical strain techniques are random by nature and can be either natural or induced. Accordingly, the entire genome of the parental strain is probed in contrast to modern era site-directed genome engineering, such as CRISPR, affecting exclusively specific target genes. A benefit hereof is that improved complex phenotypes which may be governed by the interaction between multiple genes can be identified. In absence of thorough understanding of the parental strain genome, such types of improved complex phenotypes are unlikely to be identified by specific genomic substitutions. Moreover, strains developed by the classical strain improvement approach are considered non-genetically modified organisms (GMO) which negates the commercial barriers caused by the strict GMO regulations of, for example, the EU.

The derivative Bacillus strains disclosed herein are obtained by mutagenesis of a parental strain with desired traits through an adaptive laboratory evolution (ALE) campaign. ALE is used to improve strain performance and stability through (accumulation of) beneficial mutations. With this approach, microbial strains are cultivated under specified growth conditions for prolonged periods of time, in the range from weeks to months or years, with regular passages of the cells in fresh growth media. Directed by the set of growth conditions, the microbial strain will adapt and accumulate beneficial mutations as part of a natural evolution scheme. Notably, the ALE campaign also spurs genome wide mutations that aid the fitness and growth of the microbial strain.

Accordingly, mutagenesis may be accomplished by an ALE campaign wherein a parental Bacillus strain is exposed to a stress condition, such as elevated saline levels, to purposefully introduce mutations in the genome. Therefore, the evolution campaign is referred to as Salt Tolerance Adaptive Laboratory Evolution (SALTY-ALE).

The ability of Bacillus strains to form biofilm plays an important role for root colonization that will give the bacterial inoculant a competitive advantage for populating the rhizosphere and shield plants from abiotic stress by trapping water in the biofilm. The ability to form this type of organized structure promotes long-term adhesion of the bacteria to the liquid or solid interphases and enhances survival of the bacteria under conditions of sub-optimal pH and/or nutrient deficiency. Thus, biofilm formation is an advantageous characteristic of bacterial biostimulants.

To obtain derivative Bacillus strains with improved biofilm formation and/or root colonization, the SALTY-ALE campaign was followed by a secondary evolutionary step, wherein biofilm formation was encouraged, and advantageous derivative Bacillus strains were identified and isolated.

Accordingly, herein are disclosed derivative Bacillus strains obtained from parental Bacillus strains which through mutagenesis have acquired increased salt tolerance and improved biofilm formation and/or root colonization. This combination of traits enhances the ability of the derivative Bacillus strain to promote plant fitness under conditions of abiotic stress, such as high salinity or drought.

Thus, an aspect of the present invention relates to a derivative Bacillus velezensis strain, or variant thereof, with increased salt tolerance and root colonization compared to a parental strain of Bacillus velezensis deposited as DSM34004 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

An embodiment of the present invention relates to the derivative Bacillus velezensis strain, wherein the derivative Bacillus velezensis strain has improved biofilm formation and/or root colonization.

It is to be understood that derivative Bacillus strains with identical or similar phenotypes also forms part of the invention. These may be obtained by the method described herein or by further evolution of derivative strains disclosed herein producing new variants with identical or similar phenotypes. Such strains may be said to have all of the identifying characteristics of the derivative strains disclosed herein. Accordingly, strains sharing all identifying characteristics can have different non-identical genomic sequences. The identifying characteristics may include, but is not limited to, the increased salt tolerance, biofilm formation and/or root colonization.

Thus, an embodiment of the present invention relates to a derivative Bacillus strain, or variant thereof with all the identifying characteristics thereof, with increased salt tolerance compared, biofilm formation and/or root colonization to a parental strain of Bacillus.

The main cause of increased soil salinity is the application of irrigation water which carries along soluble salt which stay in the soil after water has evaporated and transpired. The state of high salinity is compounded in dry periods or even drought. Typical salts found in irrigation water include sodium chloride (NaCl), sodium sulphate (Na₂SO₄), sodium bicarbonate (NaHCO₃), magnesium sulphate (MgSO₄), calcium sulphate (CaSO₄), calcium chloride (CaCl₂)), potassium chloride (KCl), and potassium sulphate (K₂SO₄). In particular, high concentration of sodium chloride can be detrimental to plant health.

An embodiment of the present invention relates to the derivative Bacillus strain as described herein, wherein said derivative Bacillus velezensis strain has increased salt tolerance compared to said parental strain of Bacillus velezensis, when cultured under the same conditions.

Parental strains of Bacillus may have varying baseline salt tolerance, i.e. the resistance to elevated salt concentrations before they are adapted through an evolution campaign. However, parental Bacillus strains with a lower baseline salt tolerance may be a desirable starting point because they have other desired traits, such as enhanced biostimulant properties. Accordingly, for some applications it is worth assessing the improved salt tolerance as a fold-change compared to baseline salt tolerance.

Thus, an embodiment of the present invention relates to the derivative Bacillus velezensis strain as described herein, wherein said derivative Bacillus velezensis strain has a salt tolerance which is at least 2.5 fold increased compared to a parental strain of Bacillus velezensis, such as at least 3 fold increased, such as 3.5 fold increased, such as 4 fold increased compared to a parental strain of Bacillus velezensis.

For other applications it may be anticipated that the derivative Bacillus strain is exposed to a given concentration of salt over extended periods of time or frequent enough to warrant a target absolute salt tolerance.

Accordingly, an embodiment of the present invention relates to the derivative Bacillus velezensis strain as described herein, wherein said derivative Bacillus velezensis strain has a salt tolerance of at least 0.8 M NaCl, such as at least 0.9 M NaCl, such as at least 1.0 M NaCl, such as at least 1.1 M NaCl, such as at least 1.2 M NaCl, such as at least 1.3 M NaCl, such as at least 1.4 M NaCl, such as at least 1.5 M NaCl.

Another embodiment of the present invention relates to the derivative Bacillus velezensis strain as described herein, wherein said salt tolerance is defined by the half-maximal inhibitory concentration (IC50).

The IC50 value can be determined by inoculating the derivative Bacillus velezensis culture in liquid growth medium supplemented with a pre-determined concentration of NaCl followed by incubation. Optical density measurement of cultures incubated at different NaCl concentrations can be collated and plotted as a growth curve, which may subsequently be converted to inhibition curves from which the IC50 value can be read from the fit.

For some Bacillus strains small additions of NaCl enhances the growth which will cause an offset of the inhibition curve to negative absolute inhibitory values at low NaCl concentration. In these cases, an effective concentration (EC) may be utilized if desired. The EC50 is the concentration of NaCl that gives half-maximal response.

Therefore, an embodiment of the present invention relates to the derivative Bacillus strain as described herein, wherein said salt tolerance is defined by the EC50.

The salt tolerance of the derivative Bacillus velezensis strain is to be distinguished from the actual salt levels in the habitat of the plant, e.g. the soil. The salt levels in soil are less than the salt levels under which salt tolerance of the derivative Bacillus velezensis strain is measured since plants cannot survive at these elevated salt levels. However, supplying derivative Bacillus strains with increased salt tolerance as described herein benefit the health of plants growing in habitats with increased salt levels.

The derivative Bacillus strains are in addition to the ability to proliferate under high salt stress selected for their ability to colonize the roots of plants. A measure of how effective the derivative Bacillus strains are in colonizing the roots is the preference for populating the roots compared the overall growth.

Thus, an embodiment of the present invention relates to the derivative Bacillus velezensis strain as described herein, wherein root colonization is determined as the ratio of cells attached to the seedling roots to the total number of bacterial cells.

Without being bound by theory, it is contemplated that this ability to colonize the roots is correlated with the ability to form biofilm. The derivative Bacillus velezensis strains identified herein have developed a significantly improved ability to colonize the roots of plants. Thus, the derivative Bacillus velezensis strains has a markedly higher preference for populating the plant roots compared to their parental strain.

Therefore, an embodiment of the present invention relates to the derivative Bacillus velezensis strain as described herein, wherein root colonization of said derivative Bacillus velezensis is increased at least two-fold, such as at least three-fold, such as at least four-fold, such as at least five-fold, such as at least six-fold, such as at least seven-fold, compared to said parental strain of Bacillus velezensis.

Another embodiment of the present invention relates to the derivative Bacillus velezensis strain as described herein, wherein root colonization of said derivative Bacillus velezensis is increased by at least 50%, such as at least 100%, such as at least 200%, such as at least 300%, such as at least 400%, such as at least 500%, such as at least 600%, such as at least 700% compared to said parental strain of Bacillus velezensis.

A further embodiment of the present invention relates to the derivative Bacillus velezensis strain as described herein, wherein at least 10%, such as at least 15%, such as at least 20%, such as at least 25%, such as at least 30%, such as at least 35% of the derivative Bacillus velezensis cells attach to the seedling roots in respect of the total number of bacterial cells.

The evolution campaign promotes introduction of random mutations to a parental strain followed by screening and selection of improved variants. Thus, the derivative Bacillus velezensis strains comprise genomes that are distinct from their parental strain and in turn result in improved phenotypes, such as increased salt tolerance and improved root colonization.

Therefore, an embodiment of the present invention relates to the derivative Bacillus velezensis strain as described herein, wherein said derivative Bacillus velezensis strain is genetically distinct from said parental strain of Bacillus velezensis.

Another embodiment of the present invention relates to the derivative Bacillus velezensis strain as described herein, wherein said derivative Bacillus velezensis strain comprises one or more mutations compared to said parental strain of Bacillus velezensis.

A mutation is an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift. During the SALTY-ALE campaign and the subsequent screening and evaluation of the evolved Bacillus velezensis strains several target genes were revealed as particularly promising for producing derivative Bacillus velezensis strains with improved properties.

An embodiment of the present invention relates to the derivative Bacillus velezensis strain as described herein, wherein said one or more mutations are deletion(s), substitution(s), insertion(s) and/or frame shifts.

Another embodiment of the present invention relates to the derivative Bacillus velezensis strain as described herein, wherein the one or more mutations are located in one or more genes of said parental strain of Bacillus velezensis selected from the group consisting of:
comP encoding sensor histidine kinase ComP;
dnaA-1 encoding chromosomal replication inhibitor protein;
yjcG encoding putative phosphoesterase YjcG;
sinR encoding HTH-type transcriptional regulator SinR; and
yceD-2 encoding general stress protein.

A further embodiment of the present invention relates to the derivative Bacillus velezensis strain as described herein, wherein said one or more mutations are located in one or more genes of said parental strain of Bacillus velezensis comprising a nucleic acid sequence selected from the group consisting of SEQ ID NO: 1 (comP), SEQ ID NO: 2 (dnaA-1), SEQ ID NO:3 (yjcG), SEQ ID NO:4 (sinR), SEQ ID NO:5 (yceD-2), and combinations thereof.

A still further embodiment of the present invention relates to the derivative Bacillus velezensis strain as described herein, wherein said nucleic acid sequence of the derivative Bacillus velezensis strain has at least 90% sequence identity to the nucleic acid sequence of the parental strain of Bacillus velezensis, such as at least 95% sequence identity, such as least 98% sequence identity, such as at least 99% sequence identity to the nucleic acid sequence of the parental strain of Bacillus velezensis.

It was found that mutations in the sinR gene resulted in derivative Bacillus velezensis strains with improved root colonization potential. The sinR gene encodes the main regulator of biofilm genes, such as the eps operon required for exopolysaccharide synthesis and export, the tapA-sipW-tasA operon encoding the protein components of the biofilm. Without being bound by theory, it is contemplated herein that mutations in the sinR gene lead to Bacillus velezensis with advantageous phenotypes of improved biofilm formation and increased root colonization.

Thus, an embodiment of the present invention relates to the derivative Bacillus velezensis strain as described herein, wherein said one or more mutations compared to said parental strain of Bacillus at least comprises one or more mutations in sinR.

Other favorable mutations that resulted in increased salt tolerance were identified during the SALTY-ALE campaign, hereunder mutations affecting the sensory histidine kinase, ComP, as well as in a phosphoesterase, YjcG, and a chromosomal replication initiator protein, DnaA-1.

Accordingly, an embodiment of the present invention relates to the derivative Bacillus velezensis strain as described herein, wherein said one or more mutations are located in one or more genes of said parental strain of Bacillus velezensis selected from the group consisting of comP, dnaA-1, yjcG, and combinations thereof.

Another embodiment of the present invention relates to the derivative Bacillus velezensis strain as described herein, wherein said one or more mutations are located in one or more genes of said parental strain of Bacillus velezensis selected from the group consisting of comP, dnaA-1, yjcG, sinR and combinations thereof.

A further embodiment of the present invention relates to the derivative Bacillus velezensis strain as described herein, wherein said one or more mutations compared to said parental strain of Bacillus at least comprises one or more mutations in comP, dnaA-1, yjcG, and sinR.

A still further embodiment of the present invention relates to the derivative Bacillus velezensis strain as described herein, wherein said one or more mutations compared to said parental strain of Bacillus at least comprises one or more mutations in sinR and/or dnaA-1.

A preferred embodiment of the present invention relates to the derivative Bacillus velezensis strain as described herein, wherein said one or more mutations compared to said parental strain of Bacillus at least comprises one or more mutations in dnaA-1.

The mutations promoted by the dual step campaign produce derivative Bacillus strains with new phenotypes. Favorable phenotypes have resulted in increased salt tolerance and improved root colonization properties. These phenotypes are a result of changes to the proteome of the evolved Bacillus strains.

Therefore, an embodiment of the present invention relates to the derivative Bacillus velezensis strain as described herein, wherein said one or more mutations lead to one or more modifications in one or more proteins of said parental strain of Bacillus velezensis comprising an amino acid sequence selected from the group consisting of SEQ ID NO: 6 (comP), SEQ ID NO: 7 (dnaA-1), SEQ ID NO: 8 (yjcG), SEQ ID NO: 9 (sinR), SEQ ID NO: 10 (yceD-2), and combinations thereof.

Another embodiment of the present invention relates to the derivative Bacillus velezensis strain as described herein, wherein said one or more mutations lead to one or more modifications compared to said parental strain of Bacillus velezensis selected from the group consisting of YjcG-delta (458-514), ComP-Y372C, DnaA-1-R262Q, DnaA-1-A135T, DnaA-1-L155V, DnaA-1-A283S, DnaA-1-A283G, sinR-Y11D, and combinations thereof.

A further embodiment of the present invention relates to the derivative Bacillus velezensis strain as described herein, wherein said one or more mutations lead to one or more modifications compared to said parental strain of Bacillus velezensis comprises at least YjcG-delta (458-514), ComP-Y372C, and DnaA-1-R262Q.

A still further embodiment of the present invention relates to the derivative Bacillus velezensis strain as described herein, wherein said one or more mutations lead to one or more modifications compared to said parental strain of Bacillus velezensis comprises at least YjcG-delta (458-514), ComP-Y372C, DnaA-1-R262Q, and SinR-Y11D.

Yet another embodiment of the present invention relates to the derivative Bacillus velezensis strain as described herein, wherein the genome and/or proteome of said derivative Bacillus velezensis strain is at least 95%, such as at least 98%, such as at least 99%, such as at least 99.5%, such as at least 99.8%, such as at least 99.9% identical to the genome and/or proteome of said parental strain of Bacillus velezensis.

Particular advantageous derivative Bacillus strains include those with high salt tolerance and good root colonization and biostimulant properties.

A preferred embodiment of the present invention relates to the derivative Bacillus velezensis strain as described herein, wherein said derivative Bacillus velezensis strain is deposited as DSM34319 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 30 Jun. 2022.

Gram-positive bacteria, such as Bacillus, are capable of forming spores, typically in the form of intracellular spores called endospores, as a surviving mechanism. These endospores are very retractile and thick-walled structures that constitute the most dormant form of bacteria as they exhibit minimal metabolism, respiration and enzyme production. Such bacterial spores are highly resistant to temperature fluctuations, chemical agents, UV radiation, pH gradients, drought and nutrition depletion. As the surrounding environment favors bacterial proliferation, the bacterial spores will germinate back into vegetative cells, i.e. an active bacterial cell undergoing metabolism.

Accordingly, spore-forming bacteria are preferred in the present context as they possess the ability to lay dormant if conditions in the field does not favor survival. Thus, this risk of losing the derivative Bacillus strain after application to the plant or the soil is reduced for spore-forming bacteria.

Therefore, an embodiment of the present invention relates to the derivative Bacillus velezensis strain as described herein, wherein the derivative Bacillus velezensis strain is in the form of spores or vegetative cells, preferably spores.

Derivative Bacillus velezensis strains with increased salt tolerance and increased root colonization properties may be generated by an evolution campaign as described herein. The methodology comprises at least three steps, starting with identification and provision of a parental Bacillus velezensis strain that has desired traits. The parental strain may be selected based on criteria such as its baseline salt tolerance, biofilm formation capabilities, root colonization properties and/or biostimulant properties. The selected parental strain is then cultivated under conditions of high salinity to select favourable mutations that will allow the Bacillus velezensis to survive and/or reveal increased fitness under this abiotic stress. From a population of evolved Bacillus velezensis several individual derivatives with increased salt tolerance can be obtained, studied and selected.

Thus, an aspect of the present invention relates to a method for preparing a derivative Bacillus velezensis strain with increased salt tolerance and/or root colonization compared to a parental strain of Bacillus velezensis as described herein, said method comprising the steps of:
(i) providing a parental strain of Bacillus velezensis
(ii) growing said parental strain of Bacillus velezensis under conditions of high salinity to prepare a pool of derivative Bacillus velezensis strains comprising one or more mutations, and
(iii) selecting a derivative Bacillus velezensis strain with increased salt tolerance and/or root colonization from said pool of derivative Bacillus velezensis strains.

Preferably, a derivative Bacillus strain with both increased salt tolerance and improved root colonization is selected from the pool of evolved bacterial candidates. Thus, a preferred embodiment of the present invention relates to a method for preparing a derivative Bacillus velezensis strain with increased salt tolerance and root colonization compared to a parental strain of Bacillus velezensis as described herein, said method comprising the steps of:
(i) providing a parental strain of Bacillus velezensis
(ii) growing said parental strain of Bacillus velezensis under conditions of high salinity to prepare a pool of derivative Bacillus velezensis strains comprising one or more mutations, and
(iii) selecting a derivative Bacillus velezensis strain with increased salt tolerance and root colonization from said pool of derivative Bacillus velezensis strains.

At the end of step (ii), i.e. growing of the parental strain of Bacillus under conditions of high salinity, an evolved population of Bacillus velezensis has been prepared. This population will comprise a pool of different bacterial cells (strains) which can be selected and isolated in the subsequent step (iii). The selection step may be executed in different manners. Preferably, the evolved population is streaked or spread on a solid growth medium, such as an agar plate, and colonies are manually selected therefrom.

Thus, an embodiment of the present invention relates to the method as described herein, wherein the selection step (iii) comprises providing the evolved population of step (ii) to a solid growth medium.

Several strategies may be employed for selecting derivative Bacillus velezensis strains with beneficial phenotypes. The salt concentration may be adjusted to make the selection pressure more or less harsh. Typically, very high salt concentration will decrease the number of colonies making screening faster but also less explorative/diverse. Alternatively, the evolved population may be provided to solid growth media comprising different concentrations of salt to increase the diversity of the colonies from which candidate derivative Bacillus velezensis strains are selected. Thus, colonies of derivative Bacillus velezensis strains may be selected either from solid growth media of a single salt concentration or of multiple salt concentrations.

An embodiment of the present invention relates to the method as described herein, wherein the selection step (iii) comprises providing the evolved population of step (ii) to solid growth media of different salt concentration.

The evolved population obtained from step (ii) may be applied directly to the solid growth medium in the selection step (iii) or undergo treatment prior to the selection step (iii).

Thus, an embodiment of the present invention relates to the method as described herein, wherein the evolved population of step (ii) is plated directly on the solid growth medium in the selection step (iii).

Another embodiment of the present invention relates to the method as described herein, wherein the evolved population of step (ii) is subjected to a washing step before plating on the solid growth medium in the selection step (iii).

Without being bound by theory, it is contemplated that it is advantageous to pass the evolved population through a rich medium to eliminate the salt stress prior to plating and selecting the evolved population on the solid growth medium. Relaxation of the evolved population can eliminate strains that appear to be improved based solely on an induced salt stress response and can ultimately result in more stable strains.

Therefore, an embodiment of the present invention relates to the method as described herein, wherein the evolved population is passed through a rich medium before plating on the solid growth medium in the selection step (iii).

Another embodiment of the present invention relates to the method as described herein, wherein the rich medium is fresh liquid medium, such as fresh LB medium.

Without being bound by theory, it is contemplated that the ability of bacterial strains to form biofilm is closely related to their capacity to efficiently colonize the roots of plants. To improve identification of derivative Bacillus velezensis strains with increased root colonization properties, candidate derivative Bacillus velezensis strains with improved salt tolerance may therefore be subjected to extended growth on LB growth medium enriched with glycerol and manganese (LBGM) to induce biofilm formation.

Thus, an embodiment of the present invention relates to the method as described herein further comprising a step of subjecting the pool of derivative Bacillus velezensis strains to extended growth on LB growth medium enriched with glycerol and manganese (LBGM) prior to step (iii).

Derivative Bacillus velezensis strain candidates should be able to proliferate during the selection step (iii). Proliferation may be determined by visual inspection or more quantitatively by optical density (OD) or equivalent measurements. The selection step (iii) may also comprise assessment of biofilm formation and/or root attachment to identify the most promising derivative Bacillus velezensis candidates.

Therefore, an embodiment of the present invention relates to the method as described herein, wherein said selection of step (iii) comprises quantification of the proliferation of said pool of derivative Bacillus velezensis strains under the growth conditions of step (ii).

Another embodiment of the present invention relates to the method as described herein, wherein quantification of proliferation is performed by optical density measurement.

Yet another embodiment of the present invention relates to the method as described herein, wherein said selection of step (iii) comprises assessment of biofilm formation and/or root attachment of said pool of derivative Bacillus velezensis strains.

A further embodiment of the present invention relates to the method as described herein, wherein assessment of root attachment of said pool of derivative Bacillus velezensis strains comprises determination of the ratio of cells attached to the seedling roots to the total number of bacterial cells.

A still further embodiment of the present invention relates to the method as described herein, wherein assessment of biofilm formation is performed by visual inspection.

An even further embodiment of the present invention to the method as described herein, wherein assessment of biofilm formation is performed by staining followed by spectrophotometric measurement.

The SALTY-ALE campaign is by nature random, and it is not possible a priori to know the properties of the evolved population. An advantage of this type of random mutagenesis evolution is that a large spectrum of genes and the interrelations are probed as opposed to site-directed approaches wherein only few, but specific genes are mutated. In this manner it is possible to identify even complex phenotypes. At the selection step, the derivative Bacillus velezensis strains are selected only based on the evolutionary parameter of high salinity, preferably in comparison to the parental Bacillus velezensis strain.

Therefore, an embodiment of the present invention relates to the method as described herein, wherein said conditions of high salinity is defined as a growth medium with a NaCl concentration of at least 0.8 M NaCl, such as at least 0.9 M NaCl, such as at least 1.0 M NaCl, such as at least 1.1 M NaCl, such as at least 1.2 M NaCl, such as at least 1.3 M NaCl, such as at least 1.4 M NaCl, such as at least 1.5 M NaCl.

Another embodiment of the present invention relates to the method as described herein, wherein said conditions of high salinity is obtained by incremental increase of the salinity level.

A further embodiment of the present invention relates to the method as described herein, wherein said incremental increase of the salinity level starts from a growth medium with no salt supplemented to the standard medium.

Parental Bacillus velezensis strains may be chosen based on their baseline salt tolerance, i.e. prior to any salt tolerance evolution, biofilm formation capabilities, root colonization properties and/or biostimulant properties. Herein, a particular parental Bacillus velezensis strain has been found to be an advantageous outset for developing improved derivative Bacillus velezensis strains for boosting plant health.

Thus, an embodiment of the present invention relates to the method as described herein, wherein said parental strain of Bacillus velezensis is deposited as DSM34004 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

The methods described herein may be carried out to produce and select derivative Bacillus velezensis strains with increased salt tolerance and root colonization in a systematic and optimised manner. The derivative Bacillus velezensis strains may advantageously be used as part of compositions with beneficial properties for plant health under conditions of abiotic stress, such as high salinity or drought.

Accordingly, an aspect of the present invention relates to a derivative Bacillus velezensis strain with increased salt tolerance and root colonization compared to a parental strain of Bacillus velezensis obtainable by the method as described herein.

Another aspect of the present invention relates to a composition comprising the derivative Bacillus velezensis strain with increased salt tolerance and root colonization compared to a parental strain of Bacillus velezensis as described herein.

Derivative Bacillus velezensis strains are preferably spore-forming since endospores have significantly enhanced ability to withstand any stress condition. Spores of the derivative Bacillus velezensis strains will therefore increase the robustness and longevity of the composition, especially when applied under harsh conditions.

Therefore, an embodiment of the present invention relates to the composition as described herein, wherein said composition comprises spores of said derivative Bacillus velezensis strain.

The compositions may comprise additional ingredients that improve the physical or functional properties of the composition. Thus, additional ingredients may benefit e.g. stability, deliverability, wetting, penetration or retention. Additional active ingredient beyond the derivative Bacillus velezensis strain can afford the composition with dual mode of action and include standard ingredients that are typically used in formulations of plant growth promoting agents, plant biostimulants, or biopesticides.

Thus, an embodiment of the present invention relates to the composition as described herein, wherein said composition further comprises one or more agrochemically acceptable excipients, carriers, surfactants, dispersants and yeast extracts.

Another embodiment of the present invention relates to the composition as described herein, wherein the agrochemically acceptable excipients or carriers are selected from the group consisting of maltodextrine, silicon dioxide, modified zeolite, kaolinite, lignin, starch, chitosan, and calcium carbonate.

A still further embodiment of the present invention relates to the composition as described herein, wherein said composition further comprises one or more active ingredients.

An even further embodiment of the present invention relates to the composition as described herein, wherein said one or more active ingredients are selected from the group consisting of an insecticide, fungicide, nematicide, bactericide, herbicide, plant extract, plant growth regulator, a plant growth stimulator, and a fertilizer.

Yet another embodiment of the present invention relates to the composition as described herein, wherein said one or more active ingredients are of microbial, biological or chemical origin.

Another embodiment of the present invention relates to the composition as described herein, wherein said insecticide is selected from the group consisting of pyrethroids, bifenthrin, tefluthrin, zeta-cypermethrin, organophosphates, chlorethoxyphos, chlorpyrifos, tebupirimfos, cyfluthrin, fiproles, fipronil, nicotinoids, and clothianidin, and combinations thereof.

Still another embodiment of the present invention relates to the composition as described herein, wherein said fungicide is selected from the group consisting of fluopyram plus tebuconazole, chlorothalonil, thiophanate-methyl, prothioconazole, and copper hydroxide, and combinations thereof.

The composition may comprise more than one strain of bacteria. Such consortia of bacteria can work in synergy to increase the beneficial effects of the recipient plant. The bacterial consortium may comprise e.g. bacteria that functions as biopesticide and/or bacteria that acts as biofertilizers.

Therefore, an embodiment of the present invention relates to the composition as described herein, wherein said one or more active ingredients are selected from second strains of bacteria different from said derivative Bacillus velezensis strain with increased salt tolerance and root colonization compared to a parental strain of Bacillus velezensis.

Another embodiment of the present invention relates to the composition as described herein, wherein said second strain of bacteria is a biostimulant strain, preferably a biostimulant Bacillus strain.

A further embodiment of the present invention relates to the composition as described herein, wherein said second strain of bacteria is of a species selected from the group consisting of Bacillus velezensis, Bacillus paralicheniformis, Bacillus amyloliquefaciens, and Bacillus subtilis.

The composition can be provided in a variety of different forms including, but not limited to, a liquid, an oil dispersion, a dust, a dry wettable powder, a spreadable granule, or a dry wettable granule. More specifically the composition may for example be an emulsion concentrate (EC), a suspension concentrate (SC), a water dispersible granule (WG), an emulsifiable granule (EG), a water-in-oil emulsion (EO), an oil-in-water emulsion (EW), a micro-emulsion (ME), an oil dispersion (OD), an oil miscible liquid (OL), a soluble concentrate (SL), a dispersible concentrate (DC), or a wettable powder (WP).

Thus, an embodiment of the present invention relates to the composition as described herein, wherein said composition is a form selected from the group consisting of a liquid, a wettable powder, a granule, a spreadable granule, a wettable granule, a microencapsulation, and a planting matrix.

Coating polymers are useful for providing solid entities with an outer shell that can be protective or add extra properties to the entity it is coating. Coating polymers may be part of liquid formulations which are then applied to another entity. For compositions in solid form, such as powders or granules, a coating polymer may therefore form an outer shell on the particles. For compositions in liquid form, a coating polymer may make the composition suitable for coating of other entities, such as seeds or plants.

Therefore, an embodiment of the present invention relates to the composition as described herein, wherein said composition further comprises a coating polymer.

A further embodiment of the present invention relates to the composition as described herein, wherein said composition is a liquid formulation.

An aspect of the present invention relates to a plant or seed coated with a composition as described herein.

The composition is not limited to coating a particular type of seed or plant. It is contemplated that any seed or plant may benefit from coating with the composition, in particular seeds or plants that will be exposed to abiotic stress, such as high salinity or drought. For example, by coating seeds with the composition germination can be improved for seeds sown in soil which are or will become subject abiotic stress.

The plant seed can include, but is not limited to, the seed of monocots, dicots, cereals, corn, sweet corn, popcorn, seed corn, silage corn, field corn, rice, wheat, barley, sorghum, asparagus, berry, blueberry, blackberry, raspberry, loganberry, huckleberry, cranberry, gooseberry, elderberry, currant, cane berry, bush berry brassica vegetables, broccoli, cabbage, cauliflower, brussels sprouts, collards, kale, mustard greens, kohlrabi, bulb vegetables, onion, garlic, shallots, citrus, orange, grapefruit, lemon, tangerine, tangelo, pomelo, fruiting vegetables, pepper, tomato, eggplant, ground cherry, tomatillo, okra, grape, herbs/spices, cucurbit vegetables, cucumber, cantaloupe, melon, muskmelon, squash, watermelon, pumpkin, leafy vegetables, lettuce, celery, spinach, parsley, radicchio, legumes/vegetables (succulent and dried beans and peas), beans, green beans, snap beans, shell beans, soybeans, dry beans, garbanzo beans, lima beans, peas, chick peas, split peas, lentils, oil seed crops, canola, castor, coconut, cotton, flax, oil palm, olive, peanut, rapeseed, safflower, sesame, sunflower, soybean, pome fruit, apple, crabapple, pear, quince, mayhaw, root/tuber and corm vegetables, carrot, potato, sweet potato, beets, ginger, horseradish, radish, ginseng, turnip, stone fruit, apricot, cherry, nectarine, peach, plum, prune, strawberry, tree nuts, almond, pistachio, pecan, walnut, filberts, chestnut, cashew, beechnut, butternut, macadamia, kiwi, banana, agave, ornamental plants, poinsettia, hardwood cuttings, oak, maple, sugarcane, sugarbeet, grass, or turf grass.

An embodiment of the present invention relates to the plant or seed as described herein, wherein said composition is present in an amount suitable to benefit plant growth.

Another embodiment of the present invention relates to the plant or seed as described herein, wherein said composition comprises a number of vegetative cells or spores of the derivative Bacillus velezensis strain from about 1.0×10² CFU/seed to about 1.0×10¹¹ CFU/seed, such as about 1.0×10³ CFU/seed to about 1.0×10¹⁰ CFU/seed, such as about 1.0×10⁴ CFU/seed to about 1.0×10⁹ CFU/seed.

A further embodiment of the present invention relates to the plant as described herein, wherein said composition comprises a number of vegetative cells or spores of the Bacillus strain from about 1.0×10⁴ CFU/g of roots to about 1.0×10¹⁰ CFU/g of roots, such as about 1.0×10⁵ CFU/g of roots to about 1.0×10⁹ CFU/g of roots.

The Bacillus velezensis strains and compositions comprising them can advantageously being utilized for increasing the resistance of plants against abiotic stress, such as high salinity or drought. Application may be directly to the plant, i.e. foliar application, or into the habitat of the plant.

Thus, an aspect of the present invention relates to a method of increasing resistance of a plant against a condition of abiotic stress, said method comprising administering a derivative Bacillus velezensis strain with increased salt tolerance and root colonization compared to a parental strain of Bacillus velezensis or a composition as described herein to the plant, to a part of the plant and/or to the habitat of the plant.

An embodiment of the present invention relates to the method as described herein, wherein said condition of abiotic stress is high salinity, drought and/or nutrient deficiency.

A preferred embodiment of the present invention relates to the method as described herein, wherein said condition of abiotic stress is high salinity.

The salinity level in the habitat of the plant will affect its ability to grow. High salinity levels will typically cause a significant decrease in plant mass and growth speed and under severe circumstances lead to plant death. Normal NaCl levels in soil are in the range of approx. 10-40 mM.

Thus, an embodiment of the present invention relates to the method as described herein, wherein high salinity is defined by a NaCl concentration in the habitat of the plant of more than about 50 mM, such as more than about 60 mM, such as more than about 70 mM, such as more than about 80 mM, such as more than about 90 mM, such as more than about 100 mM, such as more than about 110 mM, such as more than about 120 mM, such as more than about 130 mM, such as more than about 140 mM, such as more than about 150 mM.

Another embodiment of the present invention relates to the method as described herein, wherein high salinity is defined by a NaCl concentration in the habitat of the plant in the range of about 50 mM to about 150 mM, such as about 100 mM to about 150 mM.

Soil salinity may be measured by the electrical conductivity (EC) of the of the soil given in the unit dS/m. 10 mM NaCl has an EC close to 1 dS/m. 100 mM NaCl has an EC close to 9.8 dS/m.

The method of increasing resistance of a plant against a condition of abiotic stress is not limited to any particular species since most plants will be affected by abiotic stress, such as high salinity or drought. In particular, biofilm-forming bacteria can mitigate such stress conditions by trapping water for the benefit of the plants. Typically, the method is mainly relevant for agriculture, because relatively small improvements in yield can make a great difference in an industrial setting. Moreover, the prospect of being able to improve yield in a climate-friendly manner is attractive and preferred over traditional agrochemicals that cause widespread ecological damage.

An embodiment of the present invention relates to the method as described herein, wherein the plant is selected from the group consisting of a crop, a monocotyledonous plant, a dicotyledonous plant, a tree, a herb, a bush, a grass, a vine, a fern, and a moss.

Other plants that may benefit from the method include, but are not limited to, main crops, fruticulture, horticulture and floriculture. Main crops may be, but is not limited to, sugar cane, coffee, soybeans, cotton, corn, potatoes, tomatoes, tobacco, banana, rice, wheat, avocado, pineapple, squash, cacao, coconut, oats, onion, lettuce, beet, carrot, cassava, beans, sunflower, pepper, turnip, apple, strawberry, okra, radish and onion. Fruticulture includes, but are not limited to, citrus, grape, guava, papaya, fig, peach, plum and loquat. Floriculture may be rose, chrysanthemum, lisianthus, gerbera, amaryllis, begonia and celosia.

An embodiment of the present invention relates to the method as described herein, wherein the plant is selected from the group consisting of wheat, barley, oats, small cereal grains, corn, rice, sugar cane, soybean, potato, carrot, coffee and banana.

An embodiment of the present invention relates to the method as described herein, wherein the part of the plant is selected from the group consisting of a seed, fruit, root, stem, leaf, corm, tuber, bulb and rhizome.

Inoculation of plants with the derivative Bacillus velezensis strain or composition described herein benefit plant health under field conditions since the bacteria are capable of efficiently and preferentially colonizing the plant, more specifically the roots. Therefore, it is preferred to apply the derivative Bacillus strain or composition to the habitat of the plant.

Accordingly, an embodiment of the present invention relates to the method as described herein, wherein said derivative Bacillus velezensis strain or said composition is applied to the habitat of the plant.

Another embodiment of the present invention relates to the method as described herein, wherein the habitat of the plant is a liquid or soil, preferably soil.

The Bacillus strain may also be used for combatting abiotic stress of green algae.

Accordingly, an aspect of the present invention relates to a method of increasing resistance of a green algae against a condition of abiotic stress, said method comprising administering a derivative Bacillus strain with increased salt tolerance compared to a parental strain of Bacillus or a composition as described herein to the green algae and/or to the habitat of the green algae.

For reduction of abiotic stress in green algae, the habitat may be a liquid, such as water.

The derivative Bacillus velezensis strain or composition may be provided to the plant, to a part of the plant and/or to the habitat of the plant. Depending on the form of the derivative Bacillus velezensis strain or composition, application can be performed by dusting or spraying. Dusting, as used herein, refers to distribution of dry, finely powdered or granular compositions, typically after mixing with an inert carrier. Such application can typically be implemented in most agricultural settings without the need for investment in additional equipment.

Yet another embodiment of the present invention relates to the method as described herein, wherein said derivative Bacillus velezensis strain or said composition is applied to the habitat of the plant by spraying or dusting.

Application of the derivative Bacillus velezensis strain or composition may be performed as a preparatory measure prior to sowing or planting, or when the plant is already growing in the habitat, e.g. on the field.

Thus, an embodiment of the present invention relates to the method as described herein, wherein the derivative Bacillus velezensis strain or said composition is applied before, during or after the plant or part of the plant comes into contact with the habitat.

The derivative Bacillus velezensis strain or composition may be provided to the soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

An embodiment of the present invention relates to the method as described herein, wherein said derivative Bacillus velezensis strain or said composition is applied at least about 365 days, such as at least about 200 days, such as at least about 100 days, such as at least about 30 days, such as at least about 10 days, such at least about 5 days, such as at least 1 day, such as at least about 12 hours, such as at least about 5 hours, such as at least about 1 hour, before the plant or part of the plant comes into contact with the habitat.

Another embodiment of the present invention relates to the method as described herein, wherein said derivative Bacillus velezensis strain or said composition is applied to said seed at a rate of about 1×10⁴ to about 1×10⁸ cfu per seed, such as 1×10⁵ to about 5×10⁷ cfu per seed.

A further embodiment of the present invention relates to the method as described herein, wherein said derivative Bacillus velezensis strain or said composition is applied at a rate of about 1×10⁷ to about 1×10¹⁴ cfu per acre, such as about 1×10⁸ to about 1×10¹³ cfu per acre, such as about 1×10⁹ to about 1×10² cfu per acre.

Prior to application of the derivative Bacillus velezensis strain or composition, the habitat may be analysed to identify any condition of abiotic stress. This order of action is preferred to avoid wasting material if no action is needed. Traditional means for measuring e.g. salinity levels in soil can be applied.

Therefore, an embodiment of the present invention relates to the method as described herein, wherein said condition of abiotic stress is identified prior to application of said derivative Bacillus velezensis strain or said composition.

Another embodiment of the present invention relates to the method as described herein, wherein said condition of abiotic stress is identified by measuring the total soluble salts by evaporation of a soil water extract (TSS) or the electrical conductivity (EC).

A further embodiment of the present invention relates to the method as described herein, wherein TSS or EC is measured of a 1:5 distilled water:soil dilution or a saturated paste extract.

The derivative Bacillus velezensis strain or composition is capable of colonizing the root system of plants and provide a biostimulatory effect. The specific biostimulatory effect may vary between plants and can be a combination of several beneficial effects at an early stage of the plant life, e.g. as a seedling, and at a later stage of the plant development.

Accordingly, an embodiment of the present invention relates to the method as described herein, wherein increasing said resistance of a plant against a condition of abiotic stress facilitates improved seedling vigor, improved root development, improved plant growth, improved plant health, increased yield, improved appearance, or a combination thereof.

Another aspect of the present invention relates to use of a derivative Bacillus velezensis strain with increased salt tolerance and root colonization compared to a parental strain of Bacillus velezensis or a composition as described herein as a biostimulant, a growth promoter and/or to increase resistance of a plant against a condition of abiotic stress.

An embodiment of the present invention relates to the use as described herein, wherein said condition of abiotic stress is high salinity, drought and/or nutrient deficiency, preferably high salinity.

Another embodiment of the present invention relates to the use as described herein, wherein increasing said resistance of a plant against a condition of abiotic stress facilitates improved seedling vigor, improved root development, improved plant growth, improved plant health, increased yield, improved appearance, reduced pathogenic infection, or a combination thereof.

The derivative Bacillus velezensis strain, composition or coated seeds may conveniently be provided as a kit for easy application. The kit may comprise other active ingredients for mixing prior to distribution to the plants or their habitat.

Therefore, an aspect of the present invention relates to a kit comprising:
(i) a derivative Bacillus velezensis strain with increased salt tolerance and root colonization compared to a parental strain of Bacillus velezensis, a composition, or coated plant seeds as described herein;
(ii) a container; and
(iii) optionally, instructions for use.

An embodiment of the present invention relates to the kit as described herein, wherein the kit further comprises one or more active ingredients selected from the group consisting of an insecticide, fungicide, nematicide, bactericide, herbicide, plant extract, plant growth regulator, a plant growth stimulator, and fertilizer.

Another embodiment of the present invention relates to the kit as described herein, wherein said derivative Bacillus velezensis strain and said one or more active ingredients are provided in separate compartments in the container.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the derivative strains and all its features, which may readily be part of the part of the method or use for promoting health and growth of a plant under conditions of abiotic stress, such as high salinity. Embodiments and features of the present invention are also outlined in the following items.

### Items

Y1. A derivative Bacillus velezensis strain, or variant thereof, with increased salt tolerance and root colonization compared to a parental strain of Bacillus velezensis deposited as DSM34004 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

Y2. The derivative Bacillus strain according to item Y1, wherein said derivative Bacillus velezensis strain has increased salt tolerance compared to said parental strain of Bacillus velezensis, when cultured under the same conditions.

Y3. The derivative Bacillus velezensis strain according to any one of items Y1 or Y2, wherein said derivative Bacillus velezensis strain has a salt tolerance which is at least 2.5 fold increased compared to a parental strain of Bacillus velezensis, such as at least 3 fold increased, such as 3.5 fold increased, such as 4 fold increased compared to a parental strain of Bacillus velezensis.

Y4. The derivative Bacillus velezensis strain according to any one of the preceding items, wherein said derivative Bacillus velezensis strain has a salt tolerance of at least 0.8 M NaCl, such as at least 0.9 M NaCl, such as at least 1.0 M NaCl, such as at least 1.1 M NaCl, such as at least 1.2 M NaCl, such as at least 1.3 M NaCl, such as at least 1.4 M NaCl, such as at least 1.5 M NaCl.

Y5. The derivative Bacillus velezensis strain according to any one of the preceding items, wherein said salt tolerance is defined by the half-maximal inhibitory concentration (IC50).

Y6. The derivative Bacillus velezensis strain according to any one of the preceding items, wherein root colonization is determined as the ratio of cells attached to the seedling roots to the total number of bacterial cells.

Y7. The derivative Bacillus velezensis strain according to any one of the preceding items, wherein root colonization of said derivative Bacillus velezensis is increased at least two-fold, such as at least three-fold, such as at least four-fold, such as at least five-fold, such as at least six-fold, such as at least seven-fold, compared to said parental strain of Bacillus velezensis.

Y8. The derivative Bacillus velezensis strain according to any one of the preceding items, wherein at least 10%, such as at least 15%, such as at least 20%, such as at least 25%, such as at least 30%, such as at least 35%, of the derivative Bacillus velezensis cells attach to the seedling roots in respect of the total number of bacterial cells.

Y9. The derivative Bacillus velezensis strain according to any one of the preceding items, wherein said derivative Bacillus velezensis strain is genetically distinct from said parental strain of Bacillus velezensis.

Y10. The derivative Bacillus velezensis strain according to any one of the preceding items, wherein said derivative Bacillus velezensis strain comprises one or more mutations compared to said parental strain of Bacillus velezensis.

Y11. The derivative Bacillus velezensis strain according to item Y10, wherein said one or more mutations are deletion(s), substitution(s), insertion(s) and/or frame shifts.

Y12. The derivative Bacillus velezensis strain according to any one of items Y10 or Y11, wherein the one or more mutations are located in one or more genes of said parental strain of Bacillus velezensis selected from the group consisting of:
comP encoding sensor histidine kinase ComP;
dnaA-1 encoding chromosomal replication inhibitor protein;
yjcG encoding putative phosphoesterase YjcG;
sinR encoding HTH-type transcriptional regulator SinR; and
yceD-2 encoding general stress protein.

Y13. The derivative Bacillus velezensis strain according to any one of items Y10-Y12, wherein said one or more mutations are located in one or more genes of said parental strain of Bacillus velezensis comprising a nucleic acid sequence selected from the group consisting of SEQ ID NO: 1 (comP), SEQ ID NO:2 (dnaA-1), SEQ ID NO:3 (yjcG), SEQ ID NO: 4 (sinR), SEQ ID NO:5 (yceD-2), and combinations thereof.

Y14. The derivative Bacillus velezensis strain according to any one of items Y10-Y13, wherein said one or more mutations are located in one or more genes of said parental strain of Bacillus velezensis selected from the group consisting of comP, dnaA-1, yjcG, sinR and combinations thereof.

Y15. The derivative Bacillus velezensis strain according to any one of items Y10-Y14, wherein said one or more mutations lead to one or more modifications in one or more proteins of said parental strain of Bacillus velezensis comprising an amino acid sequence selected from the group consisting of SEQ ID NO: 6 (comP), SEQ ID NO:7 (dnaA-1), SEQ ID NO: 8 (yjcG), SEQ ID NO:9 (sinR), SEQ ID NO:10 (yceD-2), and combinations thereof.

Y16. The derivative Bacillus velezensis strain according to any one of items Y10-Y15, wherein said one or more mutations lead to one or more modifications compared to said parental strain of Bacillus velezensis selected from the group consisting of YjcG-delta (458-514), ComP-Y372C, DnaA-1-R262Q, DnaA-1-A135T, DnaA-1-L155V, DnaA-1-A283S, DnaA-1-A283G, SinR-Y11D, and combinations thereof.

Y17. The derivative Bacillus velezensis strain according to any one of the preceding items, wherein said derivative Bacillus velezensis strain is deposited as DSM34319 at Deutsche Sammlung von Mikroorganismen und Zellkulturen GmbH (DSMZ), Inhoffenstr.

7B, D-38124 Braunschweig, Germany, by Chr. Hansen A/S, Hørsholm, Denmark on 30 Jun. 2022.

Y18. The derivative Bacillus velezensis strain according to any one of the preceding items, wherein the derivative Bacillus velezensis strain is in the form of spores or vegetative cells, preferably spores.

X1. A method for preparing a derivative Bacillus velezensis strain with increased salt tolerance and/or root colonization compared to a parental strain of Bacillus velezensis according to any one of the preceding items, said method comprising the steps of:
(i) providing a parental strain of Bacillus velezensis
(ii) growing said parental strain of Bacillus velezensis under conditions of high salinity to prepare a pool of derivative Bacillus velezensis strains comprising one or more mutations, and
(iii) selecting a derivative Bacillus velezensis strain with increased salt tolerance and/or root colonization from said pool of derivative Bacillus velezensis strains.

X2. The method according to item X1 further comprising a step of subjecting the pool of derivative Bacillus velezensis strains to extended growth on LB growth medium enriched with glycerol and manganese (LBGM) prior to step (iii).

X3. The method according to any one of items X1 or X2, wherein said selection of step (iii) comprises quantification of the proliferation of said pool of derivative Bacillus velezensis strains under the growth conditions of step (ii).

X4. The method according to item X3, wherein quantification of proliferation is performed by optical density measurement.

X5. The method according to any one of item X1-X4, wherein said selection of step (iii) comprises assessment of biofilm formation and/or root attachment of said pool of derivative Bacillus velezensis strains.

X6. The method according to item X5, wherein assessment of root attachment of said pool of derivative Bacillus velezensis strains comprises determination of the ratio of cells attached to the seedling roots to the total number of bacterial cells.

X7. The method according to any one of items X5 or X6, wherein assessment of biofilm formation is performed by visual inspection.

X8. The method according to any one of items X1-X7, wherein said conditions of high salinity is defined as a growth medium with a NaCl concentration of at least 0.8 M NaCl, such as at least 0.9 M NaCl, such as at least 1.0 M NaCl, such as at least 1.1 M NaCl, such as at least 1.2 M NaCl, such as at least 1.3 M NaCl, such as at least 1.4 M NaCl, such as at least 1.5 M NaCl.

X9. The method according to any one of items X1-X8, wherein said conditions of high salinity is obtained by incremental increase of the salinity level.

X10. The method according to any one of items X1-X9, wherein said parental strain of Bacillus velezensis is deposited as DSM34004 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

P1. A derivative Bacillus velezensis strain with increased salt tolerance and root colonization compared to a parental strain of Bacillus velezensis obtainable by the method according to any one of items X1-X10.

Q1. A composition comprising the derivative Bacillus velezensis strain with increased salt tolerance and root colonization compared to a parental strain of Bacillus velezensis according to any one of items Y1-Y18 or P1.

Q2. The composition according to item Q1, wherein said composition comprises spores of said derivative Bacillus velezensis strain.

Q3. The composition according to any one of items Q1 or Q2, wherein said composition further comprises one or more agrochemically acceptable excipients, carriers, surfactants, dispersants and yeast extracts.

Q4. The composition according to any one of items Q1-Q3, wherein said composition further comprises one or more active ingredients.

Q5. The composition according to item Q4, wherein said one or more active ingredients are selected from the group consisting of an insecticide, fungicide, nematicide, bactericide, herbicide, plant extract, plant growth regulator, a plant growth stimulator, and a fertilizer.

Q6. The composition according to any one of items Q4 or Q5, wherein said one or more active ingredients are of microbial, biological or chemical origin.

Q7. The composition according to items Q5 or Q6, wherein said insecticide is selected from the group consisting of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, and clothianidin, and combinations thereof.

Q8. The composition according to any one of items Q5-Q7, wherein said fungicide is selected from the group consisting of fluopyram plus tebuconazole, chlorothalonil, thiophanate-methyl, prothioconazole, and copper hydroxide, and combinations thereof.

Q9. The composition according to any one of items Q4-Q8, wherein said one or more active ingredients are selected from second strains of bacteria different from said derivative Bacillus velezensis strain with increased salt tolerance and root colonization compared to a parental strain of Bacillus velezensis.

Q10. The composition according to item Q9, wherein said second strain of bacteria is a biostimulant strain, preferably a biostimulant Bacillus strain.

Q11. The composition according to any one of items Q9 or Q10, wherein said second strain of bacteria is of a species selected from the group consisting of Bacillus velezensis, Bacillus paralicheniformis, Bacillus amyloliquefaciens, and Bacillus subtilis.

Q12. The composition according to any one of items Q1-Q11, wherein said composition is a form selected from the group consisting of a liquid, a wettable powder, a granule, a spreadable granule, a wettable granule, a microencapsulation, and a planting matrix.

Q13. The composition according to any one of items Q1-Q12, wherein said composition further comprises a coating polymer.

Q14. The composition according to any one of items Q1-Q13, wherein said composition is a liquid formulation.

R1. A plant or seed coated with a composition according to any one of items Q1-Q14.

R2. The plant or seed according to item R1, wherein said composition is present in an amount suitable to benefit plant growth.

R3. The plant or seed according to any one of items R1 or R2, wherein said composition comprises a number of vegetative cells or spores of the derivative Bacillus velezensis strain from about 1.0×10² CFU/seed to about 1.0×10¹¹ CFU/seed, such as about 1.0×10³ CFU/seed to about 1.0×10¹⁰ CFU/seed, such as about 1.0×10⁴ CFU/seed to about 1.0×10⁹ CFU/seed.

Z1. A method of increasing resistance of a plant against a condition of abiotic stress, said method comprising administering a derivative Bacillus velezensis strain with increased salt tolerance and root colonization compared to a parental strain of Bacillus velezensis according to any one of items Y1-Y18 or P1 or a composition according to any one of items Q1-Q14 to the plant, to a part of the plant and/or to the habitat of the plant.

Z2. The method according to item Z1, wherein said condition of abiotic stress is high salinity, drought and/or nutrient deficiency.

Z3. The method according to any one of items Z1 or Z2, wherein said condition of abiotic stress is high salinity.

Z4. The method according to any one of items Z1-Z3, wherein high salinity is defined by a NaCl concentration in the habitat of the plant of more than about 50 mM, such as more than about 60 mM, such as more than about 70 mM, such as more than about 80 mM, such as more than about 90 mM, such as more than about 100 mM, such as more than about 110 mM, such as more than about 120 mM, such as more than about 130 mM, such as more than about 140 mM, such as more than about 150 mM.

Z5. The method according to any one of items Z1-Z4, wherein high salinity is defined by a NaCl concentration in the habitat of the plant in the range of about 50 mM to about 150 mM, such as about 100 mM to about 150 mM.

Z6. The method according to any one of items Z1-Z5, wherein the plant is selected from the group consisting of a crop, a monocotyledonous plant, a dicotyledonous plant, a tree, a herb, a bush, a grass, a vine, a fern, and a moss.

Z7. The method according to any one of items Z1-Z6, wherein the plant is selected from the group consisting of wheat, barley, oats, small cereal grains, corn, rice, sugar cane, soybean, potato, carrot, coffee and banana.

Z8. The method according to any one of items Z1-Z7, wherein the part of the plant is selected from the group consisting of a seed, fruit, root, stem, leaf, corm, tuber, bulb and rhizome.

Z9. The method according to any one of items Z1-Z8, wherein said derivative Bacillus velezensis strain or said composition is applied to the habitat of the plant.

Z10. The method according to any one of items Z1-29, wherein said derivative Bacillus velezensis strain or said composition is applied to the habitat of the plant by spraying or dusting.

Z11. The method according to any one of items Z1-Z10, wherein the habitat of the plant is a liquid or soil, preferably soil.

Z12. The method according to any one of items Z1-Z11, wherein the derivative Bacillus velezensis strain or said composition is applied before, during or after the plant or part of the plant comes into contact with the habitat.

Z13. The method according to any one of items Z1-Z12, wherein said condition of abiotic stress is identified prior to application of said derivative Bacillus velezensis strain or said composition.

Z14. The method according to any one of items Z1-Z13, wherein said condition of abiotic stress is identified by measuring the total soluble salts by evaporation of a soil water extract (TSS) or the electrical conductivity (EC).

Z15. The method according to item Z14, wherein TSS or EC is measured of a 1:5 distilled water:soil dilution or a saturated paste extract.

Z16. The method according to any one of items Z1-Z15, wherein increasing said resistance of a plant against a condition of abiotic stress facilitates improved seedling vigor, improved root development, improved plant growth, improved plant health, increased yield, improved appearance, or a combination thereof.

V1. Use of a derivative Bacillus velezensis strain with increased salt tolerance and root colonization compared to a parental strain of Bacillus velezensis according to any one of items Y1-Y18 or P1 or a composition according to any one of items Q1-Q14 as a biostimulant, a growth promoter and/or to increase resistance of a plant against a condition of abiotic stress.

V2. The use according to item V1, wherein said condition of abiotic stress is high salinity, drought and/or nutrient deficiency, preferably high salinity.

V3. The use according to any one of items V1 or V2, wherein increasing said resistance of a plant against a condition of abiotic stress facilitates improved seedling vigor, improved root development, improved plant growth, improved plant health, increased yield, improved appearance, reduced pathogenic infection, or a combination thereof.

T1. A kit comprising:
(i) a derivative Bacillus velezensis strain with increased salt tolerance and root colonization compared to a parental strain of Bacillus velezensis according to any one of items Y1-Y18 or P1, a composition according to any one of items Q1-Q14, or coated plant seeds according to any one of items R1-R3;
(ii) a container; and
(iii) optionally, instructions for use.

T2. The kit according to item T1, wherein the kit further comprises one or more active ingredients selected from the group consisting of an insecticide, fungicide, nematicide, bactericide, herbicide, plant extract, plant growth regulator, a plant growth stimulator, and fertilizer.

T3. The kit according to any one of items T1 or T2, wherein said derivative Bacillus velezensis strain and said one or more active ingredients are provided in separate compartments in the container.

The invention will now be described in further details in the following non-limiting examples.

### EXAMPLES

### Example 1: Salt Evolution Campaign and Selection of Derivative Bacillus Strains with Improved Salt Tolerance from the Evolved Populations

The parental strain Bacillus velezensis was subjected to a Salt Tolerance Adaptive Laboratory Evolution (SALTY-ALE) campaign carried out by linearly increasing NaCl concentration in the growth medium. The SALTY-ALE campaign was followed by selection of derivative strains with increased salt tolerance from the evolved populations of the SALTY-ALE campaign.

### Method

### Strain, Media and Culture Conditions of SALTY-ALE Campaign

The Bacillus strain was cultured at 30° C. (or 37° C.) in Luria-Bertani (LB) broth or on LB agar. The salt tolerance campaign was carried out in modified CSE media (10.46 g/L of MOPS, 3.3 g/L of (NH₄)₂SO₄, 0.13 g/L of KH₂PO₄, 0.267 g/L of K₂HPO₄, 3.23 mg/L of MnSO₄·4H₂O, 120 mg/L of MgSO₄·7H₂O, 22 μg/L of ammonium ferric citrate, 0.8% of potassium glutamate, 0.6% of sodium succinate, pH 7).

To make NaCl supplemented CSE media, various concentration of NaCl was added in the media by adjusting the volume of MQ-water.

To prepare a diverse set of evolved populations the parental strain Bacillus velezensis was cultivated in CSE media supplemented with 0 to 2 M of NaCl at 30° C. and 225 RPM in a Growth Profiler. Pictures of the culture plates was taken every 30 min to assess the bacterial growth over time.

### Analysis of Bacillus Growth Using a Growth Profiler Setup

Single colonies of Bacillus strains from LB-agar plates were inoculated into 400 μl of CSE media in 96 deep well-plate. Then, the plate was incubated at 30° C. and 250 RPM for 20-24 hours. After 10- to 20-fold dilutions, OD_{600 nm} was determined. The saturated overnight pre-cultures were diluted 100-fold into 250 μl of CSE media or CSE media supplemented with a pre-determined concentration of NaCl. Subsequently, the culture plates were incubated in a Growth Profiler (Enzyscreen, Heemstede, The Netherlands) at 30° C. and 250 RPM for 2-3 days with constant monitoring of the growth by scanning the plate every 30 minutes. Using the GP software (GP960Viewer version 1.0.0.4, Enzyscreen, Heemstede, The Netherlands), the pixel data of the pictures was converted into digital OD_{600 nm} values and the growth curves over time were generated.

### Relaxation Step in Rich Media and Plating

The evolved populations in the salt supplemented CSE media were first transferred in LB media to eliminate the salt stress before plating in LB agar supplemented with various concentrations of NaCl. Briefly, 500 μl of the evolved strains from the SALTY-ALE campaign were centrifuged at 5000×g for 5 min and the supernatant was discarded. The cell pellet was resuspended into 500 μl of fresh LB media. Then the resuspended cells were transferred into 4.5 ml of LB media and the culture tubes were incubated overnight at 30° C. and 250 RPM. The parental strain was also inoculated in 5 ml of LB media (as control) and also incubated overnight at 30° C. and 250 RPM. Next day, the overnight cultures were diluted in LB media to reach a final OD_{600 nm} of 1. These OD-adjusted cultures were streaked or spread on LB agar and LB agar supplemented with various concentrations of NaCl. For instance, in LB agar, LB+1M NaCl agar plates the cells were streaked out, whereas 10-25 μl of the cells were spread on LB+1.4 M NaCl, LB+1.6 M NaCl, and 25-100 μl of the cultures were plated in LB+1.89 M NaCl, LB+2 M NaCl and LB+2.2 M NaCl agar plates. The plates were incubated at 37° C. until pickable colonies were observed, i.e. after about 24-48 h.

### Results

A pool of candidate derivative strains was picked based on their ability to sustain growth on the agar plates containing high salt concentration. The pool of candidate formed the derivative strain material from which further selection with regards to biofilm formation was performed.

### Conclusion

The SALTY-ALE campaign was successfully performed to provide a pool of candidate derivative strains with improved salt tolerance from which further selection of traits could be based on.

### Example 2: Characterization of Biofilm Formation of Derivative Bacillus Strains

The pool of candidate derivative Bacillus strains of the SALTY-ALE campaign was plated on LBGM plates to identify mutants with a preferred biofilm phenotype.

### Methods

The pool of candidate derivative strains isolated from the SALTY-ALE evolution campaign were plated on LBGM plates, i.e. Lysogeny broth (LB) supplemented with glycerol and manganese. The addition of glycerol and manganese leads to the induction of biofilm gene expression via a signaling pathway involving the sensory histidine kinase KinD. Ultimately this results in a stronger colony differentiation on the plates, which makes selection of favorable phenotypes possible.

5 μl of bacterial culture in exponential growth phase were streaked on a fresh LB plate containing 0.1 mM manganese and 1% v/v glycerol (LBGM plate) and incubated over night at 37° C. Already after the over-night incubation, different colony morphologies were visible. Different areas of the streak-out were re-streaked several times on fresh LBGM plates and incubated over night at 37° C., respectively. Once the streak-outs showed a homologous colony morphology, liquid LB medium was inoculated with a single colony and cultivated until an OD_{600 nm} of about 1 was reached. Cells were then pelleted for testing of salt tolerance or genome whole sequencing.

### Results

It is desired to identify derivative Bacillus strains with highly structured and rough areas as this phenotype typically produce a biofilm that results in good root colonization properties of the Bacillus. In contrast, impaired biofilms that appear unstructured and shiny tend to perform worse in colonizing the roots of plants. The complex colony structure was evaluated by the biofilm assay on LBGM plates.

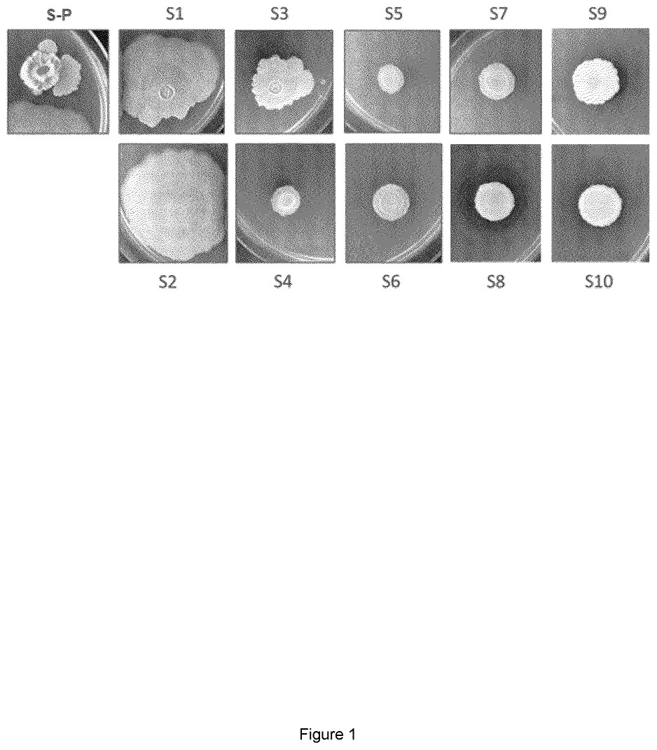

A set of 10 lead candidates (S1-S10) showed structured and rough colony appearance similar to or improved when compared to the parental strain (S-P) (FIG. 1). These lead candidates were advanced to determination of their salt tolerance and root colonization abilities.

### Conclusion

Lead candidates with favorable biofilm phenotypes were identified out of the pool of candidate derivative strains isolated from the SALTY-ALE evolution campaign.

### Example 3: Growth Curves of Selected Derivative Strains Under Conditions of Elevated Salt Concentration

The growth potential of the lead candidates (S1-S10) was determined under conditions of high salt concentration to validate the impact of the SALTY-ALE campaign.

### Methods

Growth curves of S1-S10 were obtained using the Growth Profiler setup as described in example 1. The derivative Bacillus strains were cultivated in medium supplemented with 1M NaCl.

For the Growth Profiler experiments, the starting OD600 nm of all the strains, including the parental strain, was kept within the range 0.02-0.05 to achieve comparable results.

### Results

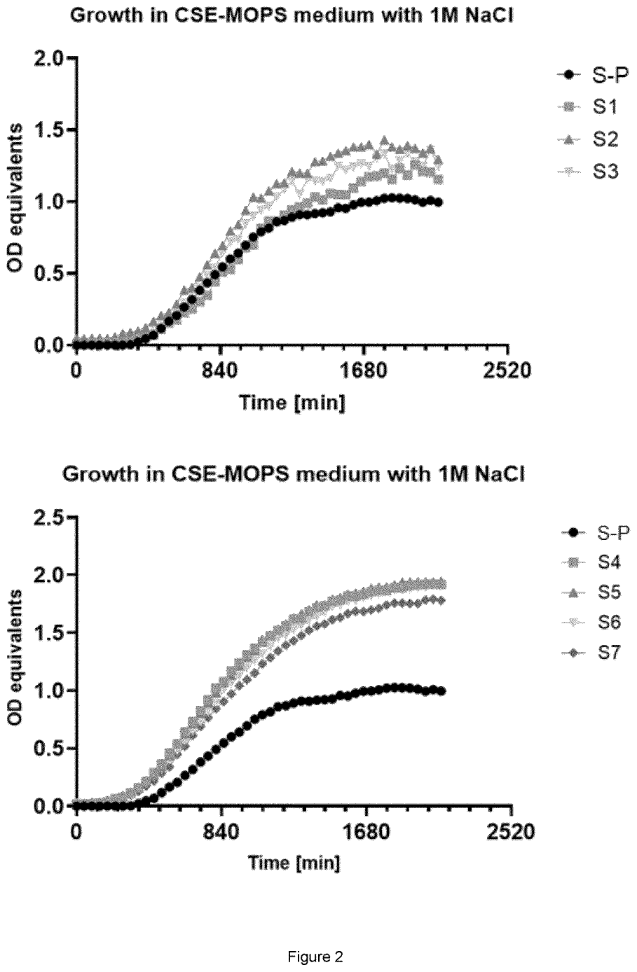

All of the lead candidates (S1-S10) showed improved proliferation at high salt concentration compared to the parental strain (S-P) (FIG. 2, data for S8-S10 not shown). All lead candidates showed improved growth rate and especially S4-S7 also showed reduced lag phase.

### Conclusion

This example demonstrates that lead candidate derivative Bacillus strains with improved biofilm formation also display improved salt tolerance compared to the parental strain.

### Example 4: Analysis of Root Colonization Potential of Derivative Bacillus Strains

The root colonization potential was determined of the lead derivative Bacillus strains.

### Methods

Arabidopsis seeds were sown in half-strength Murashige and Skoog medium supplemented with 0.5% sucrose and placed vertically in a plant growth chamber at 21° C. and light/dark period 16 hours/8 hours photoperiod for 7 days. Strains were streaked from glycerol stocks in Luria broth (LB) agar plates at 37° C. overnight. One colony was inoculated in 5 mL LB until OD 1 was reached and then the culture was diluted to OD 0.001 in root exudates surrogate medium at 30° C. overnight.

7 days old seedlings were transferred with a toothpick to a 12 well plate containing 2.5 ml of half-strength Murashige and Skoog medium and inoculated with the strain at a final OD 0.05. Inoculated seedlings were incubated in a plant growth chamber for 24 hours at 21° C. and light/dark period 16 hours/8 hours photoperiod. Bacterial growth and root attachment was assessed by serial dilution and plating of 100 μl of preferred dilution in LB agar plates and incubated at 30° C. until colony forming units (CFUs) were visible. Root attachment was assessed by moving the seedling to a 2-ml Eppendorf tube containing 1-mL of 1×PBS and sonicated (40 amp pulse for 10 seconds and 10 seconds rest, repeated 3 times).

### Results

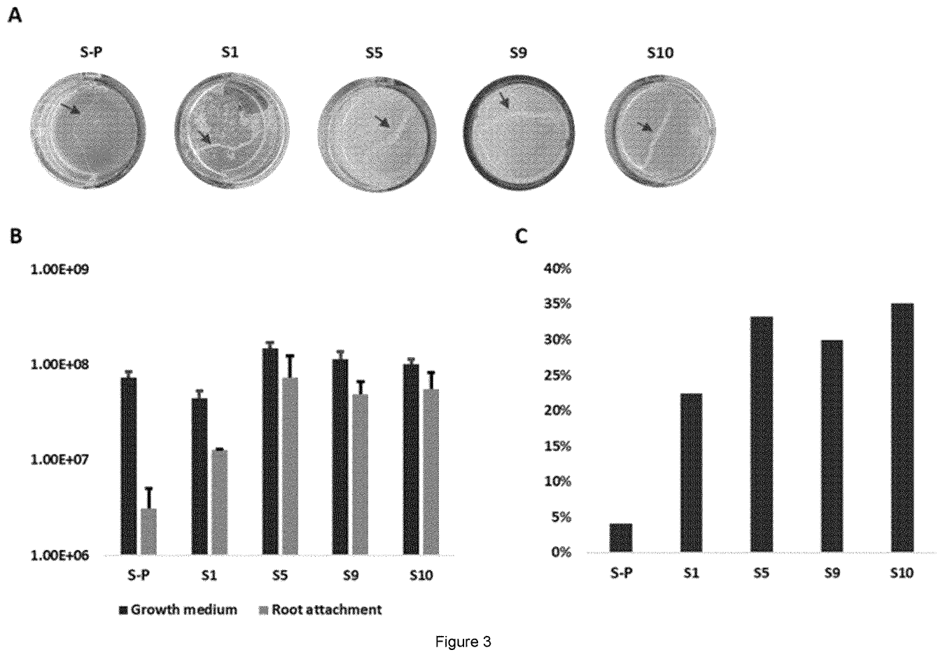

Root colonization evaluation of Arabidopsis seedlings inoculated with the lead candidate Bacillus strains showed an improvement to colonize and attach to the plant roots compared to S-P. In particular, the thickness of the roots inoculated with the lead candidate derivative Bacillus strains are higher than the ones inoculated with the parental strain S-P due to the amount of bacterial aggregated around it (FIG. 3A). Bacterial numbers at the roots are quantitatively vastly superior for the lead candidate derivative Bacillus strains compared to S-P (FIG. 3B). The percentage of cells attached to the seedling roots in respect to the total number of bacterial cells is advantageously high for the lead candidate derivative Bacillus strains with numbers exceeding 20% and up to approximately 35% compared less than 5% for the parental strain S-P.

### Conclusion

This example demonstrates that the lead candidate derivative Bacillus strains have improved root colonization capability compared to the parental strain S-P, making them advantageous for use as plant biostimulants.

### Example 5: Genetic Analysis of Derivative Bacillus Strains

The lead candidates (S1-S10) were subjected to genetic analysis to detect mutations, hereunder single nucleotide polymorphisms (SNPs), and thereby identify key target genes that can affect salt tolerance and/or biofilm formation.

### Methods

A single colony of the derivative strains were streaked in a LB plate for three rounds of purification steps. Then 5 colonies of the purified strain were inoculated in 5 ml of LB media and the tube was incubated overnight at 30° C. Next morning, 500 μl of the overnight culture was transferred in 30 ml of fresh LB media. The flask was incubated at 37° C. until the OD_{600 nm} reached about 1.0-1.5. Then, whole genome sequencing samples were prepared.

Cell pellets from 1.5 ml of 1.5 OD equivalent cultures were collected by centrifuging the cultures at 5000×g for 10 min. Then the cell pellet was submitted for gDNA isolation and whole genome sequencing.

The genomes of the derivative strains were sequenced using Illumina short-read sequencing and the resulting data was assembled to the reference genomes of the previously sequenced parental strain (S-P). The SNP calling was performed using a breseq pipeline. The breseq software is intended for microbial genomes (<10 Mb) and re-sequenced samples that are only slightly diverged from the reference sequence (<1 mutation per 1000 bp).

### Results

Whole genome sequencing of the derivative strains detected genetic changes and identified key target genes considered to play a role for improved salt tolerance and/or improved biofilm formation.

### Conclusion

Key target genes, including dnaA-1 and sinR, were identified based on genetic analysis of derivative strains with improved salt tolerance and root colonization.

### REFERENCES

Altschul et al. (1990), J. Mol. Biol., 215, 403-410

### Deposits and Expert Solution

The applicant requests that a sample of the deposited microorganisms stated in table 1 below may only be made available to an expert, until the date on which the patent is granted.

The applicant requests that the availability of the deposited microorganism referred to in Rule 33 EPC shall be effected only by the issue of a sample to an independent expert nominated by the requester (Rule 32 (1) EPC). If an expert solution has been requested, restrictions concerning the furnishing of samples apply.

The deposits were made according to the Budapest treaty on the international recognition of the deposit of microorganisms for the purposes of patent procedure at at Deutsche Sammlung von Mikroorganismen und Zellkulturen GmbH (DSMZ), Inhoffenstr. 7B, D-38124 Braunschweig, Germany.

The Budapest Treaty provides that any restriction of public access to samples of deposited biological material must be irrevocably removed as of the date of grant of the relevant patent.

TABLE 1
Deposited strains made at a depositary institution.

| Strain | Name | Accession no. | Deposit date |
|---|---|---|---|
| Bacillus velezensis | S-P | DSM34004 | 24 Aug. 2021 |
| Bacillus velezensis | S5 | DSM34319 | 30 Jun. 2022 |

1-15. (canceled)

16. A derivative Bacillus strain that exhibits increased salt tolerance and root colonization, as compared to Bacillus velezensis DSM 34004, wherein said derivative Bacillus strain is derived from Bacillus velezensis DSM 34004.

17. The derivative Bacillus strain of claim 16, wherein the salt tolerance of said derivative Bacillus strain is at least twice that of Bacillus velezensis DSM 34004.

18. The derivative Bacillus strain of claim 16, wherein the root colonization of said derivative Bacillus strain is at least twice that of Bacillus velezensis DSM 34004.

19. The derivative Bacillus strain of claim 16, wherein said derivative Bacillus strain comprises a mutation in one or more genes selected from comP, dnaA-1, yjcG, sinR, and yceD-2.

20. The derivative Bacillus strain of claim 16, wherein said derivative Bacillus strain contains a mutation in the comP gene.

21. The derivative Bacillus strain of claim 16, wherein said derivative Bacillus strain contains a mutation in the dnaA-/gene.

22. The derivative Bacillus strain of claim 16, wherein said derivative Bacillus strain contains a mutation in the yjcG gene.

23. The derivative Bacillus strain of claim 16, wherein said derivative Bacillus strain contains a mutation in the sinR gene.

24. The derivative Bacillus strain of claim 16, wherein said derivative Bacillus strain contains a mutation in the yceD-2 gene.

25. The derivative Bacillus strain of claim 16, wherein said derivative Bacillus strain comprises a nucleic acid sequence having at least 95% sequence identity to the whole genome sequence of Bacillus velezensis DSM 34004.

26. An isolated Bacillus derivative deposited as DSM 34319 at Deutsche Sammlung von Mikroorganismen und Zellkulturen GmbH (DSMZ), Inhoffenstr. 7B, D-38124 Braunschweig, Germany.

27. A composition comprising the isolated Bacillus derivative of claim 26 and an agrochemically acceptable carrier.

28. A method comprising introducing the isolated Bacillus derivative of claim 26 into a plant growth medium.

29. A method comprising contacting a plant or plant part with the isolated Bacillus derivative of claim 26.

30. A kit comprising:
the isolated Bacillus derivative of claim 26; and
instructions for introducing the isolated Bacillus derivative of claim 16 into a plant growth medium and/or for applying the isolated Bacillus derivative of claim 16 to a plant or plant part.

31. A composition comprising a plant or plant part that is at least partially coated with a composition comprising the isolated Bacillus derivative of claim 26.


SEQUENCE LISTING (10 sequences)

SEQ ID NO: 1 - DNA, 2301 bp, Bacillus velezensis
  source 1..2301: mol_type = genomic DNA
atgaagttatctaaaaatacgtattccattttattaattttactcagcttgtcttatattttctatttaacttatataaatgtaaataatttatttgtaggagccaccgcaaatgttaataacaagaatcaaatagagattaccaaagtatcagattacacaatggcttattatgctggtgttaaaaaaggtgacattgtaattaaaattaatcagaataagagcgttgaaccaaaagatttaaaacaaaatacattgaaaaacgtaagatctatgacagttgaaagatctggtgaaatagttgatctcaaaaatttaagtttattaagtgaagacaacttatttgtctatttaatacctgttatttttttctttgtatgccttagttgtattatccttattttaaaaattaataaggagcaagattcagtttcagcttttatattgatcatctttttactcactactggtatagcatatgtaagtgcgggaggttcatcaaaaggagatataataagccgatacgtaaatgtcataactttaatatccgtaccggtaaattatctcttgtttatttaccaatatatcaaagaatttggatacaagatttttaacgggaaagtttttgttgctttatatacagtccctgttattaatataatactagagttttttaggcattgctttgaaagaggagtagtggggaaagttaatttagtatcaatatctctcctttatttaatagctttatttttcattactgtaggtttaataaagtacaaaaaaacagagcaagcaaatattcttaagacttttcttttaattaatatttttgctttctcaccatttgtattcttgtatgtggcaccctacatcatttttaatgactattatgtttcttcttttttagttgcgccttttcttttattaattcctttttctttagtttatcaatttatgactaataaaatctatgaaattgattttattattgggcggttgaaatattatagtcttttagctgtcactcctactttacttattattacaatttatgatcttgtacaagatccaaactcagagttttatacgtttaaactaactttgctgacatatattattatgttggctgtgttttattcaaaagagatcttagattttcgtttccgtttaaagcgtttttctgaaaaacataattatcaggacagcatattcaagtttactcaaataattaaaggagcttcatccttaaagcaagtattcactgaacttgaatctactctacttgaagttttgatggttagtaaagtatgtgttttagaagtagaaggagacttaaatgtaaggtatttttttaatgataatgagaaaaaattatgggagccgtattctcatcaattagctgaaacgacaagcgaaattggcaaaatagttcaggtggataaagggtttttaatgaaaataggggaacggggcactaaatcatacattgtactgtgtttatcatttatgaatactccgaaactaacttgggatgagatttcttggttaaaaaccttatcattctacaccagtattacgatggagaatgtgcttaaaatcgaagaacttatgaatcatctcgaagatttaaagaaacaagagacaaacccggtatggctcaaaaagctgatgtttacgatcgaggaaaagcagcgttcggatttggcccgtgatctgcatgattcagtgctgcaggacttgatttctttgaagcgccagtgtgagctgtttctggccgattttaagaaagaggagccttgccagttagaggtgcaggacaagcttcatcagatgaatgagcagatgtcggatgtcattttgatgacccgggagacgtgccatgagcttcggcctcagctgctgtatgatttgggtctggtcaaggcggtatccaagcttgccgcacagcagcaggagcgtgcgccgtttcatatccgtctgaatacgggccggtttacggcggcgctggatttggatacgcagctgaatttgtaccgcattattcaggaatttctttcgaacgccatgaagcattcacaggcgaatgaggtgctgattatgctgatcagcattcaaaataaagtgattctgcattatgaagacgacggtgtcggattcaatcaggaagaaggcggcgggcagtcgatgagtatgggtctttcaggtattaaggagcgcgtccgtgcgcttgacggacggatgaaaatcgacacaagtgaaggaaacggttttaaagcggatattgaaatggaattgtaa

SEQ ID NO: 2 - DNA, 1341 bp, Bacillus velezensis
  source 1..1341: mol_type = genomic DNA
atggagaatatattagatctttggaatcaagctctagctcaaattgagaaaaagctaagcaaaccgagtttcgaaacttggatgaagtcgacgaaagcccattcgctgcaaggggataccttaaccatcaccgctcccaatgaatttgccagagactggctggagtccaggtacttacatctcattgcagataccatatacgaattaaccggggaagaattgagcgtcaaatttgtcattcctcaaaatcaggacgaagaaaactttttaccgaaaccgcaagtgaaaaaagcggcaaaagaggaaccgtctgattttccccaaagcatgctcaacccgaaatatacattcgacacattcgtcatcggatcgggaaaccgattcgcccatgcagcatcactggctgtggcggaagcacccgctaaagcgtacaaccctttatttatatacgggggagtcggcttaggaaaaacccacttaatgcacgcgatcggccattatgtcattgatcataatccttcagccaaagtggtgtacctgtcttctgaaaaattcacaaatgaattcatcaactcaatccgcgataataaagccgttgatttccgcaaccgctatcggaacgttgatgtacttttgatagatgatattcaatttttagccggaaaagaacaaacacaggaagagtttttccatacattcaacacgcttcatgaagaaagcaaacaaatcgtgatttccagcgacagaccgccgaaagagatcccgacccttgaagaccgcctgcgatcgcggttcgaatggggattgattacggatatcacaccgccggatctggaaacgcggattgccatcctgagaaagaaagcgaaggcggaaggccttgatattccgaacgaggtcatgctttacatcgcgaatcagattgacagcaacatccgcgaactggaaggggcattaatacgggttgtcgcctattcatccttaatcaataaagacattaacgcagacctggctgcagaagcattgaaagatattattccgtcttctaagccgaaggtcattacgataaaagagattcagcggattgtcggacagcagttcaacattaagcttgaagatttcaaagcgaaaaagcgtacgaaatccgtggcgttcccaagacaaatcgcgatgtatctttcaagagaaatgacggattcttctttgcctaaaatcggggaagaatttggcggcagagaccatacaaccgtcattcacgcccatgaaaagatttcaaagctgctgatagatgacgagcagcttcagcagcaggtgaaagaaataaaagaacttcttaaataa

SEQ ID NO: 3 - DNA, 516 bp, Bacillus velezensis
  source 1..516: mol_type = genomic DNA
atgaaatacggtattgttttatttccatcaaaagaattacaggatctcgcaaattcgtaccggaaacgctacgacccgagctacgcgctcattccgccgcatctgacggtaagagatccgtttgaagcgacagaacaggaaatcgagcaagtcgtatcacagctgagacagacggcgaaagagtcgcatccgctcgttctgaaaatcacaaaatacagctcgttttctcctgtgaataatgtgatatatatgaaagcagagccgacggaggaattaatatctctgagtgagaaatgctactcaggagcattatccggcgagccggaatacagcttcgttccgcatgtcacggtcggccagaagctgtcgtcagatgaacattccgacgtactcggacagctgaaaatgcaggacatttcccacgaagaagtcatcgaccgttttcacttgctctatcagctggataacggctcatggactgtgtacgaaacatttttattaggcggaggagaataa

SEQ ID NO: 4 - DNA, 342 bp, Bacillus velezensis
  source 1..342: mol_type = genomic DNA
atgacattgattggccagcgtattaaacaataccgaaaagaaaaaggctactcactatcagaactagctgaaaaagctggggtagcgaagtcttatttaagctcaatagaacgaaatttacagacgaacccctccattcaatttctcgaaaaagtctccgctgttctggacgtctcggttcatactttacttgatgaaaaacatgaaaccgaatacgatggtcaattagatagtgaatgggagaaattagttcgcgatgcaatgacatccggggtatcaaaaaaacaatttcgtgaatttttagattatcaaaaatggagaaaagcgcaaaaagaggagtag

SEQ ID NO: 5 - DNA, 579 bp, Bacillus velezensis
  source 1..579: mol_type = genomic DNA
atggctattcaattggcaaaaggacagcgtgttgatctgacaaaaacgaacccaggtcttacaaaagcggtgatcggcttaggctgggatacgaacaagtactccggagggcatgactttgaccttgatgcgtcggcttttcttgtcgatgcccatgacaactgtgtaaatgatcttgattttgttttctacaataacctggaacacccaagcgggggcgtcatccatacgggagacaaccggacgggcgagggcgagggagacgatgaacaaattatcgtcgacttctcaaaaatacccgcaaatattgaaaagatcggcatcaccgtcacgattcatgatgcggaagcgcgcggccaaaatttcggacaagtttcgaatgcgttcgtgcgcgtcgtcgatgaaaacagccagaatgaactgcttcgtttcgatttaggagaagacttctcgattgaaacggcggttgtcgtttgtgagctttaccgccatggggctgagtggaagtttaacgccatcggcagcgggttttccggcggtctggcatcactttgccggaattacggcttgcaggtgtaa

SEQ ID NO: 6 - protein, 766 aa, Bacillus velezensis
MKLSKNTYSILLILLSLSYIFYLTYINVNNLFVGATANVNNKNQIEITKVSDYTMAYYAGVKKGDIVIKINQNKSVEPKDLKQNTLKNVRSMTVERSGEIVDLKNLSLLSEDNLFVYLIPVIFFFVCLSCIILILKINKEQDSVSAFILIIFLLTTGIAYVSAGGSSKGDIISRYVNVITLISVPVNYLLFIYQYIKEFGYKIFNGKVFVALYTVPVINIILEFFRHCFERGVVGKVNLVSISLLYLIALFFITVGLIKYKKTEQANILKTFLLINIFAFSPFVFLYVAPYIIFNDYYVSSFLVAPFLLLIPFSLVYQFMTNKIYEIDFIIGRLKYYSLLAVTPTLLIITIYDLVQDPNSEFYTFKLTLLTYIIMLAVFYSKEILDFRFRLKRFSEKHNYQDSIFKFTQIIKGASSLKQVFTELESTLLEVLMVSKVCVLEVEGDLNVRYFFNDNEKKLWEPYSHQLAETTSEIGKIVQVDKGFLMKIGERGTKSYIVLCLSFMNTPKLTWDEISWLKTLSFYTSITMENVLKIEELMNHLEDLKKQETNPVWLKKLMFTIEEKQRSDLARDLHDSVLQDLISLKRQCELFLADFKKEEPCQLEVQDKLHQMNEQMSDVILMTRETCHELRPQLLYDLGLVKAVSKLAAQQQERAPFHIRLNTGRFTAALDLDTQLNLYRIIQEFLSNAMKHSQANEVLIMLISIQNKVILHYEDDGVGFNQEEGGGQSMSMGLSGIKERVRALDGRMKIDTSEGNGFKADIEMEL

SEQ ID NO: 7 - protein, 446 aa, Bacillus velezensis
MENILDLWNQALAQIEKKLSKPSFETWMKSTKAHSLQGDTLTITAPNEFARDWLESRYLHLIADTIYELTGEELSVKFVIPQNQDEENFLPKPQVKKAAKEEPSDFPQSMLNPKYTFDTFVIGSGNRFAHAASLAVAEAPAKAYNPLFIYGGVGLGKTHLMHAIGHYVIDHNPSAKVVYLSSEKFTNEFINSIRDNKAVDFRNRYRNVDVLLIDDIQFLAGKEQTQEEFFHTFNTLHEESKQIVISSDRPPKEIPTLEDRLRSRFEWGLITDITPPDLETRIAILRKKAKAEGLDIPNEVMLYIANQIDSNIRELEGALIRVVAYSSLINKDINADLAAEALKDIIPSSKPKVITIKEIQRIVGQQFNIKLEDFKAKKRTKSVAFPRQIAMYLSREMTDSSLPKIGEEFGGRDHTTVIHAHEKISKLLIDDEQLQQQVKEIKELLK

SEQ ID NO: 8 - protein, 171 aa, Bacillus velezensis
MKYGIVLFPSKELQDLANSYRKRYDPSYALIPPHLTVRDPFEATEQEIEQVVSQLRQTAKESHPLVLKITKYSSFSPVNNVIYMKAEPTEELISLSEKCYSGALSGEPEYSFVPHVTVGQKLSSDEHSDVLGQLKMQDISHEEVIDRFHLLYQLDNGSWTVYETFLLGGGE

SEQ ID NO: 9 - protein, 113 aa, Bacillus velezensis
MTLIGQRIKQYRKEKGYSLSELAEKAGVAKSYLSSIERNLQTNPSIQFLEKVSAVLDVSVHTLLDEKHETEYDGQLDSEWEKLVRDAMTSGVSKKQFREFLDYQKWRKAQKEE

SEQ ID NO: 10 - protein, 192 aa, Bacillus velezensis
MAIQLAKGQRVDLTKTNPGLTKAVIGLGWDTNKYSGGHDFDLDASAFLVDAHDNCVNDLDFVFYNNLEHPSGGVIHTGDNRTGEGEGDDEQIIVDFSKIPANIEKIGITVTIHDAEARGQNFGQVSNAFVRVVDENSQNELLRFDLGEDFSIETAVVVCELYRHGAEWKFNAIGSGFSGGLASLCRNYGLQV